(19) United States
(12) Patent Application Publication
(10) Pub. No.: US 2024/0090809 A1
(43) Pub. Date: Mar. 21, 2024

(54) ADAPTIVE SELECTION OF STIMULI FOR NEURO-ACTIVATION

(71) Applicant: Click Therapeutics, Inc., New York, NY (US)

(72) Inventor: Jacqueline LUTZ, New York, NY (US)

(73) Assignee: Click Therapeutics, Inc., New York, NY (US)

(21) Appl. No.: 18/368,303

(22) Filed: Sep. 14, 2023

Related U.S. Application Data
(60) Provisional application No. 63/406,850, filed on Sep. 15, 2022.

(51) Int. Cl. A61B 5/16

(52) U.S. Cl. CPC A61B 5/165

(57) ABSTRACT

Provided herein are systems and methods of associating stimuli with conditions of users. A computing system may identify, in response to presenting a first stimulus to a user, a relevance value of the first stimulus indicative of a relevance of the first stimulus with a condition of the user. The computing system may classify, responsive to the relevance value satisfying a threshold, the first stimulus as having a non-neutral reaction type associated with the condition to the user. The computing system may store, in one or more data structures, an association between the user and the first stimulus classified as having the non-neutral reaction type for presenting in a therapy session to address the condition of the user. The computing system may provide instructions for presenting the first stimulus in the therapy session to address the condition of the user.

### CROSS REFERENCES TO RELATED APPLICATIONS

The present application claims priority under 35 U.S.C. § 119(e) to U.S. Patent Provisional Application No. 63/406,850, titled “Adaptive Selection of Stimuli for Neuro-Activation,” filed Sep. 15, 2022, which is incorporated herein by reference in its entirety.

### BACKGROUND

Psychiatric disorders, such as affective disorders (e.g., major depressive disorder (MDD), post-traumatic stress disorder (PTSD), and anxiety orders) or chronic pain, may have a negative and debilitating impact on people. A multitude of intervention techniques may be used to alleviate or treat such disorders with wide variance in the therapeutic effectiveness.

### SUMMARY

Many diseases may have shared underlying cognitive and emotional impairments, which are related to certain brain systems and regions. Behavioral and psychological interventions can target specific circuits and systems of the brain. For example, depression may often arise in people suffering from challenges in emotion regulation related to altered prefrontal cortex brain connectivity to emotion processing areas (e.g., amygdala). In particular, people may struggle from different idiosyncratic, personal stressors that cause or exacerbate emotional regulation issues or pressure related to the condition. These stressors may include, for example, anxiety and stress about specific stimuli or situations, harsh self-criticism, or maladaptive behavior triggered from unique situations and stimuli, among others.

To target these idiosyncratic stressors, an application or platform running on a computing device may be used to develop and generate a training regimen for therapy sessions customized for the particular user. Modules specifying delivery stimuli for neural activation and modulation tailored for the user may be incorporated into an application on an end-user device to address the user's behavioral and psychological conditions. Each module may be configured to target additional specific systems or regions of the brain through neural activation and modulation, such as those relating to attention, memory, inhibitory control, and emotion regulation, among others. The configuration of these modules may leverage the facts that many of the impairments in these brain systems are transdiagnostic, in that alterations in such systems can underlie a variety of conditions. For example, attention bias may be present in conditions such as anxiety, addiction and pain, among other indications.

The module may include tasks for a therapy session (sometimes referred to herein as trials) in which each stimulus is repeatedly presented to a user in different variations. For instance, the user may be prompted by the application running the module to view an image and respond by pressing a button to answer a question regarding the image. These modules may allow the user to perform tasks with personally relevant stimuli of various types through the prompts, thereby stimulating multiple systems or regions of the brain. The therapy sessions as defined by the modules may use various types of stimuli, such as images, text (e.g., using words), audio, or other multimedia presentations, among others, to activate certain regions (e.g., emotion processing regions) of the brain and trigger user interaction. These tasks activate the underlying weakened or impaired brain systems or circuits, thereby alleviating the psychological condition suffered by the user.

It may be desirable that the stimuli to be delivered and the specified user interaction are personalized and related to the behavior change at issue to enable optimal neural activation and training efficacy. For instance, if the task is to address nicotine addiction, the stimuli selected may be related to nicotine addiction and may include images or text relating to nicotine, cigarettes, and smoking, among others. Furthermore, if the task is to address anxiety, the stimuli may be related to the specific anxiety that the user suffers. In the case of comorbidities, users may be presented with mixed stimuli targeting different underlying stressors or dysfunctions (e.g., addiction and anxiety stimuli).

The therapy session may be constructed around the tasks and stimuli and may be aimed at alleviating or treating idiosyncratic stressors of the particular user with respect to the psychological or behavioral condition desired to be treated. Before entering the task, the application may carry out a stimuli selection stage to facilitate identifying which stimuli best represent the user's idiosyncratic stressors. In this process, the application may prompt the user with various stimuli with a question as to how each stimulus relates to the user's experience of the condition to be addressed. The stimuli may be provided from a library of stimuli identified as non-neutral with respect to the condition (e.g., images of cigarettes for nicotine addiction condition). A subset of these stimuli may trigger a response particular to the user. The stimuli selection procedure may be performed in other ways. For instance, the user's reaction to the stimulus in the form of biomarkers, such as heart rate, heart rate variability, skin conductance, facial expressions, breathing and others may be measured using cameras, touch sensors, and other instruments. In addition, a health care provider or a therapist may also collect and record the user's response while the application presents the stimuli to the user.

From the responses, the application may determine whether the stimulus is idiosyncratic or relevant to the user. The response may include a value indicating relevance of the corresponding stimulus with the condition to be addressed for the user. The application may compare the value to a threshold demarcating neutral or non-neutral reaction types. The comparison may be used to identify stimuli that are not only personally relevant to the user and the condition to be addressed, but also has an emotional valence to the user (e.g., triggers an emotional response on the part of the user). If the relevance value is above the threshold, the application may determine that the stimulus is non-neutral and idiosyncratic for the user. The threshold may be set to adjust the difficulty of the tasks for the therapy session. In this manner, the application may be able to use the measurements from the user to objectively identify stimuli that are particularly targeted to the idiosyncratic stressors for the behavioral or psychological condition of the user.

In addition to idiosyncratic stimuli, the application may present the user in the therapy session. For certain tasks, the application may present neutral stimuli along with the non-neutral and idiosyncratic stimuli to the user to target the behavioral or psychological condition. The neutral stimuli may be stored and maintained in a library of neutral stimuli, such as a language corpus or an image database. In general, the application may select a neutral stimulus with characteristics similar to or matching characteristics of a non-neutral idiosyncratic stimulus. For instance, images for the neutral and non-neutral idiosyncratic stimuli may be similar in size, color, and intensity, among characteristics. Furthermore, words selected from a textual corpus for the neutral and non-neutral idiosyncratic stimuli may be similar in length, topic category, and frequency in use, among others. For facial expressions, the neutral and non-neutral stimuli may be generated using a morphing or transformation algorithm to create a wide intensity of different emotions. With the identification, the application may prompt the user to confirm whether the neutral stimulus is neutral for the user.

With the identification of the stimuli, the application may store an association of the non-neutral idiosyncratic and neutral stimuli with the user. For example, the application may generate a unique user identifier (UUID) for the user, and store identifications of the non-neutral idiosyncratic and neutral stimuli with the UUID on a database. During the therapy session, the application may identify the stimuli to be presented in the tasks to be carried out by the user. Upon identification, the application may present the stimulus (e.g., non-neutral idiosyncratic or neutral stimulus) along with a cue in accordance with the task. The cue may be to alter a likelihood that the user interacts with the stimulus, thereby altering the association of the stimulus with the stressor particular to the user related to the behavioral or psychological disorder. For instance, for certain tasks, the cue may be to increase the likelihood that the user interacts with the non-neutral idiosyncratic or neutral stimulus to alter the association of the stimuli in the brain of the user. Conversely, for other tasks, the cue may be to decrease the likelihood that the user interacts with the non-neutral idiosyncratic stimulus to gravitate the user away from the stimulus, thereby disassociating from the underlying stressor.

Aspects of the present disclosure are directed to a system, method, and computer-readable media for associating idiosyncratic or relevant stimuli with conditions of users. A computing system having one or more processors coupled with memory may identify, in response to presenting a first stimulus to a user, a relevance value of the first stimulus indicative of a relevance of the first stimulus with a condition of the user. The computing system may classify, responsive to the relevance value satisfying a threshold, the first stimulus as having a non-neutral idiosyncratic reaction type associated with the condition to the user. The computing system may store, in one or more data structures, an association between the user and the first stimulus classified as having the non-neutral idiosyncratic reaction type for presenting in a therapy session to address the condition of the user. The computing system may provide instructions for presenting the first stimulus in the therapy session to address the condition of the user.

In some embodiments, the computing system may select, from a plurality of stimuli classified as having a neutral reaction type, a second stimulus using a characteristic of the first stimulus. In some embodiments, the computing system may generate a set of stimuli comprising the first stimulus and the second stimulus. In some embodiments, the computing system may store the association between the user and the set of stimuli for presenting both the first stimulus and the second stimulus in the therapy session for addressing the condition of the user.

In some embodiments, the computing system may identify, from the one or more data structures, the association between a user and a set of stimuli comprising (i) the first stimulus and (ii) a second stimulus having a neutral reaction type. In some embodiments, the computing system may present, in the therapy session for the user, the first stimulus and the second stimulus, concurrent with a cue to increase a likelihood of selection of a stimulus from the set of stimuli having a target reaction type. In some embodiments, the computing system may receive a response identifying a selection by the user of a stimulus from the set of stimuli.

In some embodiments, the therapy session may include an implicit association task (TAT). In some embodiments, the computing system may present the set of stimuli to increase the likelihood of selection of the stimulus having the neutral reaction type. In some embodiments, the therapy session may include at least one of attention bias modification training (ABMT) or go/no-go training. In some embodiments, the computing system may present the cue to decrease the likelihood of selection of the stimulus having the non-neutral reaction type. In some embodiments, the therapy session may include a personal trigger memory task. In some embodiments, the computing system may present, without a cue, a plurality of stimuli including: (i) the first stimulus and (ii) a second stimulus having a neutral reaction type.

In some embodiments, the computing system may identify a plurality of delivery parameters for the user based on response data to the therapy session. In some embodiments, the computing system may identify the threshold to compare against the relevance value based on at least one of (i) a parameter of the therapy session or (ii) a relevance value of a second stimulus classified as having the non-neutral idiosyncratic reaction type for the user.

In some embodiments, the computing system may maintain, on a database, a plurality of stimuli from which to selectively select for presentation to address the condition. In some embodiments, the computing system may receive a plurality of physiological measurements of the user in response to presentation of the first stimulus to the user.

In some embodiments, the computing system may identify a first evaluation dataset for the user at a first time prior to the therapy session and a second evaluation dataset for the user at a second time subsequent to the therapy session. In some embodiments, the computing system may determine a progression for the user in addressing the condition based on the first evaluation dataset and the second evaluation dataset.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a block diagram of a system for adaptively selecting stimuli to provide sessions in accordance with an illustrative embodiment;

FIG. 2A depicts a block diagram for a process to classify stimuli for association with users in the system in accordance with an illustrative embodiment;

FIG. 2B depicts a block diagram for a process to execute sessions to deliver stimuli to users in the system in accordance with an illustrative embodiment;

FIGS. 3A-C depict block diagram of a system architecture for adaptively selecting stimuli to provide sessions in accordance with an illustrative embodiment;

FIGS. 4A-F each depict a screenshot of a user interface with a prompt regarding a degree of relevance of a presented stimulus to a user with a condition to be addressed in accordance with an illustrative embodiment;

FIGS. 5A-C each depict a screenshot of a user interface with a prompt regarding a degree of relevance of a presented stimulus to a user with a condition to be addressed in accordance with an illustrative embodiment;

FIGS. 6A-D each depict a screenshot of a user interface with a prompt regarding a degree of relevance of a presented stimulus to a user with a condition to be addressed in accordance with an illustrative embodiment;

FIG. 7A depicts a flow diagram of a method of associating stimuli with users to address conditions in accordance with an illustrative embodiment;

FIG. 7B depicts a flow diagram of a method of performing sessions to provide stimuli to users to address conditions in accordance with an illustrative embodiment; and

FIG. 8 is a block diagram of a server system and a client computer system in accordance with an illustrative embodiment.

### DETAILED DESCRIPTION

For purposes of reading the description of the various embodiments below, the following enumeration of the sections of the specification and their respective contents may be helpful:

Section A describes embodiments of systems and methods of adaptively selecting stimuli to provide sessions; and

Section B describes a network and computing environment which may be useful for practicing embodiments described herein.

### A. System and Method for Adaptive Selecting Stimuli to Provide in Sessions

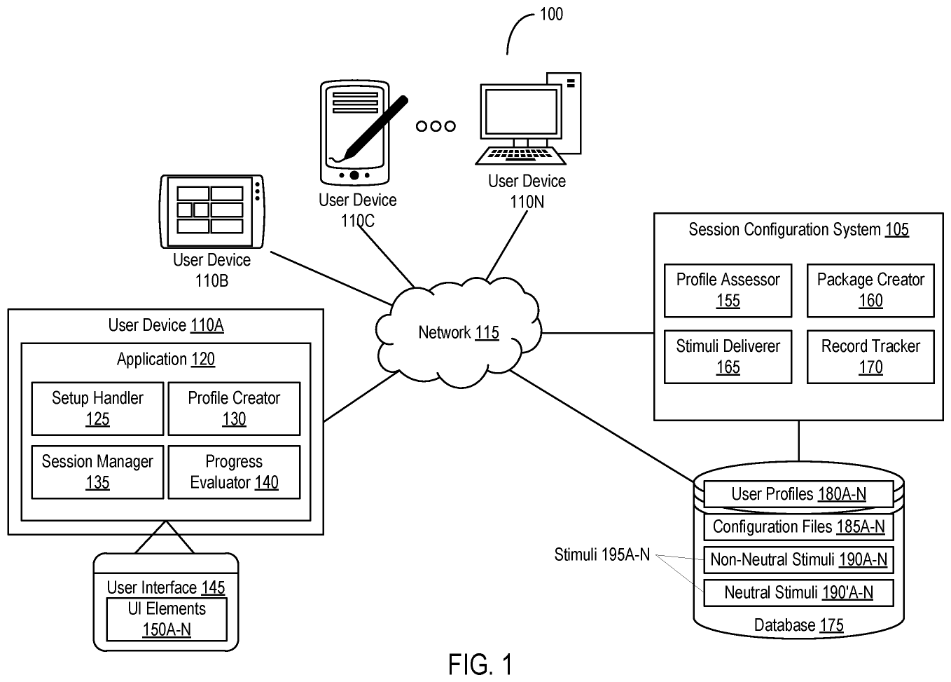

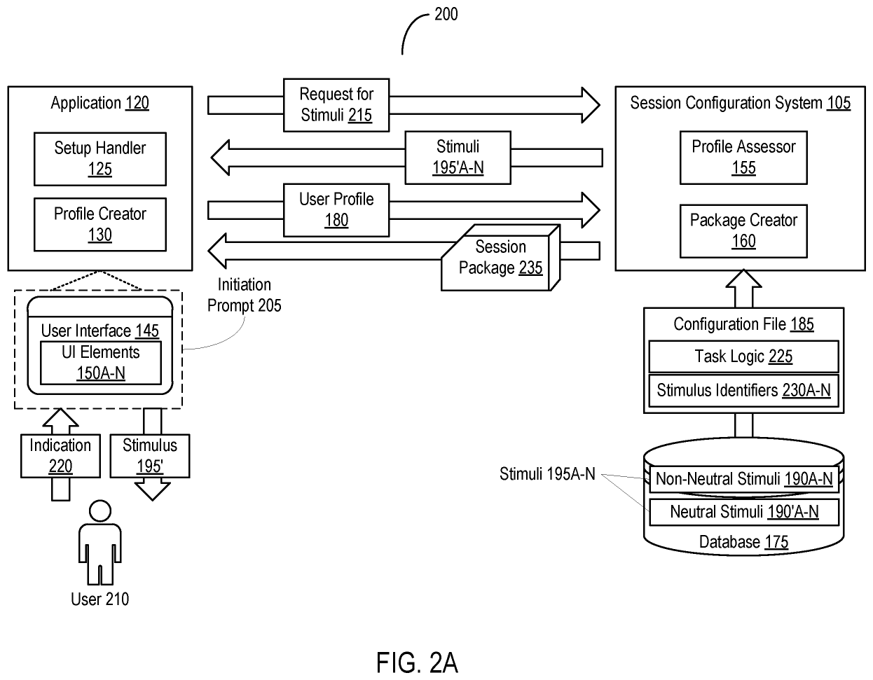

Referring now to FIG. 1, depicted is a block diagram of a system 100 for adaptively selecting stimuli to provide sessions. In overview, the system 100 may include at least one session configuration system 105 and a set of user devices 110A-N (hereinafter generally referred to as user devices 110 or clients), communicatively coupled with one another via at least one network 115. At least one user device 110 (e.g., the first user device 110A as depicted) may include at least one application 120. The application 120 may include at least one setup handler 125, at least one profile creator 130, at least one session manager 135, and at least one progress evaluator 140, among others. The application 120 may also include or provide at least one user interface 145 with one or more user interface (UI) elements 150A-N (hereinafter generally referred to as UI elements 150). The session configuration system 105 may include at least one profile assessor 155, at least one package creator 160, at least one stimuli deliverer 165, and at least one record tracker 170, among others. The session configuration system 105 may include or have access to at least one database 175. The database 175 may store, maintain, or otherwise include one or more user profiles 180A-N (hereinafter generally referred to as user profiles 180), one or more configuration files 185A-N (hereinafter generally referred to as configuration files 185), a set of non-neutral stimuli 190A-N (hereinafter generally referred to as non-neutral stimuli 190), and a set of neutral stimuli 190′A-N (hereinafter generally referred to as neutral stimuli 190′), among others. The non-neutral stimuli 190 and the neutral stimuli 190′ may be generally referred to as stimuli 195A-N (hereinafter generally referred to as stimuli 195). The functionality of the application 120 may be performed in part on the session configuration system 105.

In further detail, the application 120 executing on the user device 110 may include, present, or otherwise provide the user interface 145 including the one or more UI elements 150 to a user of the user device 110 in accordance with a configuration on the application 120. The UI elements 150 may correspond to visual components of the user interface 145, such as a command button, a text box, a check box, a radio button, a menu item, and a slider, among others. In some embodiments, the application 120 may be a digital therapeutics application and may provide a session (sometimes referred to herein as a therapy session) via the user interface 145 to address at least one condition of the user (sometimes herein referred to as a patient, person, or subject). The condition may include behavioral or psychological conditions. The psychological condition may include, for example, chronic pain (or hypersensitivity) and emotional dysregulation (e.g., post-traumatic stress disorder (PTSD), depression, addiction, or hyper-reactivity to stressors), among others. The behavioral conditions to be addressed may include, for example, smoking, alcohol overconsumption, dietary behavior, and other disorders, among others.

Each stimulus 195 (e.g., neutral stimuli 190′ and non-neutral stimuli 190) may include or correspond to information or instructions for presentation and delivery of such information. The information may be designed or configured to trigger an emotional or psychological reaction on the part of a user receiving the stimulus 195. For example, some of the stimuli 195 (e.g., the non-neutral stimuli 190) may be a picture of a gun, a diseased person, or a representation of death, and may elicit emotional or arousal response on the part of the user.

The set of stimuli 195 may be designed and constructed to account for psychiatric diseases and chronic conditions to be addressed. Many psychiatric diseases and chronic conditions may lead to indication-related stimuli being associated with a very strong emotional reaction through repeat stress or conditioning mechanisms, for instance, stimuli related to food in eating disorders; craving-related stimuli for addiction; self-criticism or shame associated with depression or other chronic conditions that impact functioning; pain-related stimuli in chronic pain conditions; emotional expressions in depression; stress-related stimuli in conditions vulnerable to stress; trauma-specific stimuli in PTSD; and negative social stimuli in schizophrenia, among others.

Each stimulus 195 (e.g., including the neutral stimuli 190′ and non-neutral stimuli 190) may be designed, constructed, or configured to activate the emotion processing system in the brain of the user, with higher intensity compared to users in healthier control groups. The emotion processing system may include the amygdala, hippocampus, and insula, among others. Within the brain, the emotion processing systems may be connected with higher order cortical systems (prefrontal cortex, attention networks, and default mode). This connectivity can alter and regulate the emotional experience.

The information for the stimulus 195 may be stored and maintained in the database 175 in the form of one or more files. Each stimulus 195 may be various types, and may include, for example, a text (e.g., including words or phrases), an image (e.g., images of faces, objects, and other items), audio, or multimedia content (e.g., including video with audio), or any combination thereof, among others. The files for the text corresponding to the stimulus 195 may be various formats, such as TXT, RTF, CSV, XML, and HTML, among others. The files for the image corresponding to the stimulus 195 may include, for example, various formats, such as BMP, TIFF, JPEG, GIF, and PNG, among others. The audio, video, or multimedia content for the stimulus 195 may include various formats, such as MPEG, WMV, and OGG, among others. The files for the stimulus 195 may also include one or more scripts (e.g., JavaScript) specifying the presentation and delivery of the information to the user.

In addition, each stimulus 195 may be associated with one or more conditions to be addressed. In some embodiments, the association may be indicated in the file for the stimulus 195 itself. In some embodiments, the association may be maintained in the database 175 separately from the files for the stimuli 195. The association may define, specify, or otherwise identify one or more conditions that the corresponding stimulus 195 is indicated to address. In some embodiments, each stimulus 195 may be associated with one or more sessions for addressing the corresponding conditions. For example, an image containing a facial expression for one non-neutral stimulus 190 may be associated with a session for a personal trigger memory task) designed to treat emotion regulation.

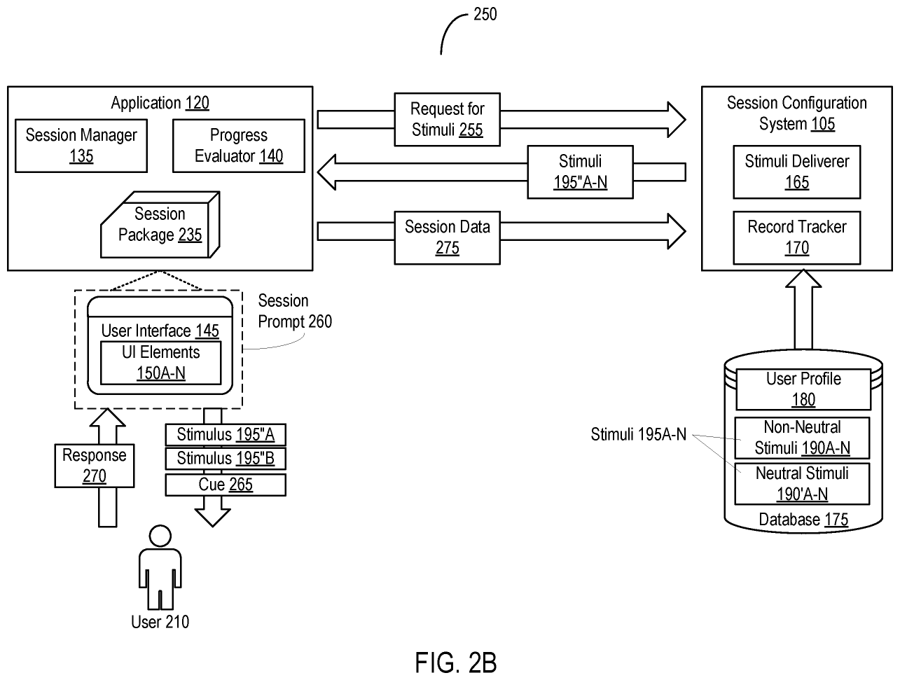

Referring now to FIG. 2A, depicted is a block diagram for a process 200 to classify stimuli for association with users in the system 100. The process 200 may include or correspond to operations performed in the system 100 to classify stimuli as non-neutral to associate with a user. Under the process 200, the setup handler 125 of the application 120 executing on the user device 110 may present or provide at least one initiation prompt 205 to a user 210 of the application 120 via the user interface 145. The initiation prompt 205 may be used by the user 210 to initialize, specify, or otherwise configure a session to address the condition of the user 210. In some embodiments, the setup handler 125 may present the initiation prompt 205 upon installation of the application 120 on the user device 110. In some embodiments, the initiation prompt 205 may be presented in response to an interaction with another UI element 150 of the application 120.

The initiation prompt 205 may identify or include a set of questions for the user 210 to identify the condition to be addressed and, subsequently, the type of stimuli for the session to address the condition. The condition may include, for example, chronic pain, emotional dysregulation, drug addiction, or dietary issues, among others. The type of stimuli preferred by the user 210 for the session may include, for example, text, images, audio, or other multimedia content, among others. The questions themselves may correspond to text, audio, or visual content on the UI elements 150 of the user interface 145. The responses to the questions may be entered or inputted by the user 210 via other UI elements 150, such as radio buttons, command buttons, text boxes, sliders, or check boxes, among others. The set of questions presented via the initiation prompt 205 may include at least one condition to be addressed (e.g., behavioral or psychological). In addition, the set of questions may include types of stimuli preferred by the user 210 in addressing the condition for the session. Using the responses from the user 210 via the user interface 145, the setup handler 125 may identify the condition to be addressed and the type of stimuli for the session to be provided to the user 210.

With the identification, the setup handler 125 may provide, transmit, or otherwise send at least one request 215 for stimuli associated with the condition of the user 210 to be addressed. The request 215 may include or identify the condition indicated by the user 210 using the initiation prompt 205 presented via the user interface 145. In some embodiments, the request 215 may identify the type of stimuli indicated by the user 210 for inclusion in the session to address or treat the condition. Using the responses, the setup handler 125 may produce, output, or otherwise generate the request 215 to identify the condition and the type of stimuli, among others. With the generation, the setup handler 125 may transmit the request 215 over the network 115 to the session configuration system 105.

The profile assessor 155 executing on the session configuration system 105 may retrieve, identify, or otherwise receive the request 215 from the application 120 running on the user device 110. Upon receipt, the profile assessor 155 may parse the request 215 to extract or identify the condition of the user 210 to be addressed. The profile assessor 155 may also identify the type of stimuli to be provided to the user 210 in addressing the condition. Using the identified condition and the type of stimuli, the profile assessor 155 may identify or select an initial set of stimuli 195′ from the database 175. The initial set may include stimuli 195′ (e.g., the non-neutral stimuli 190) to which the user 210 is to indicate whether the stimuli 195′ is relevant to the condition to be addressed. The stimuli 195′ in the initial set may correspond to the type of stimuli specified in the request 215. In some embodiments, the profile assessor 155 may access the database 175 to find, retrieve, or identify the initial set of stimuli 195′ for the condition to be addressed. The initial set of stimuli 195′ may include non-neutral stimuli 190 for addressing the condition. In some embodiments, the initial set of stimuli 195′ may include neutral stimuli 190′ associated with the condition. With the identification, the profile assessor 155 may provide, send, or otherwise transmit the initial set of stimuli 195′ to the user device 110. The setup handler 125 may in turn retrieve, identify, or receive the initial set of stimuli 195′ from the session configuration system 105.

The profile creator 130 of the application 120 executing on the user device 110 may present or provide each stimulus 195′ from the initial set to the user 210. In providing, the profile creator 130 may display, play, or otherwise present the stimulus 195′ via an input/output (I/O) device of the user device 110. For example, for a textual or image content, the profile creator 130 may render or display the stimulus 195′ via the user interface 145 as at least one of the UI elements 150. For audio content, the profile creator 130 may play the stimulus 195′ via the speakers of the user device 110. For video or multimedia content, the profile creator 130 may present the visual portion of the stimulus 195′ via the user interface 145 of the application 120 and play the audio portion of the stimulus 195′ via the speakers.

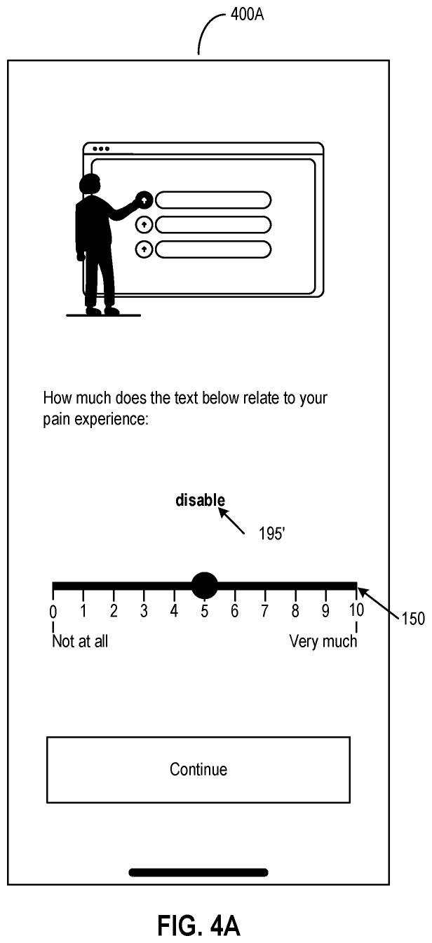

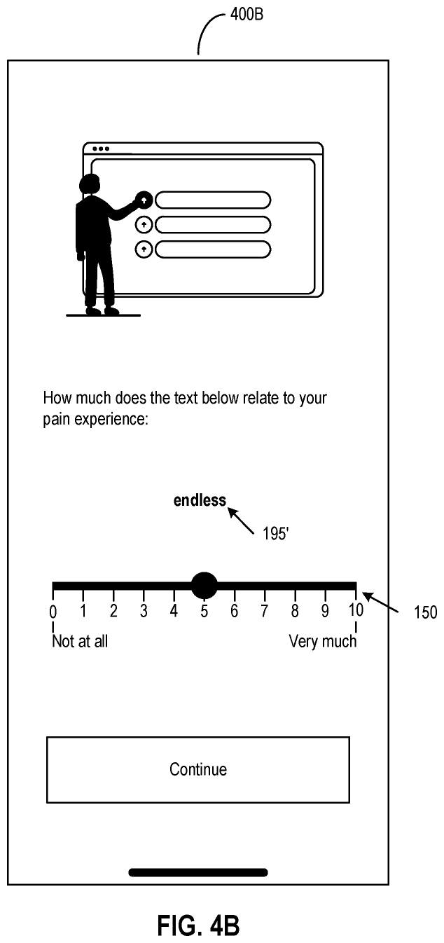

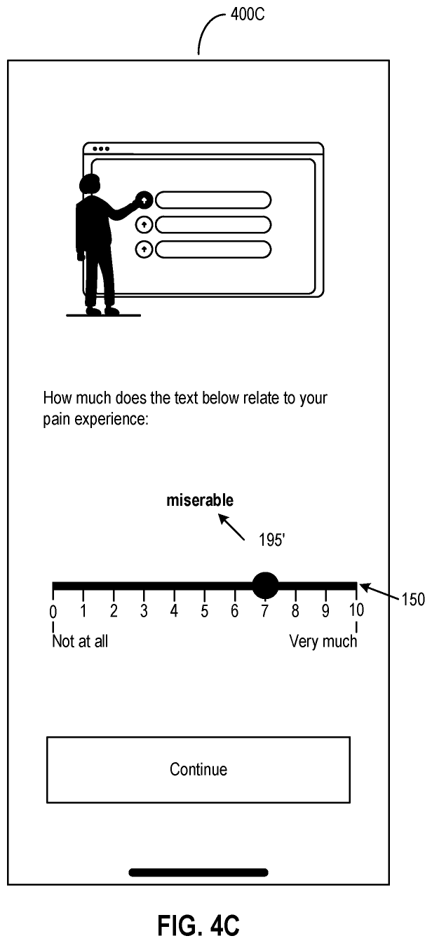

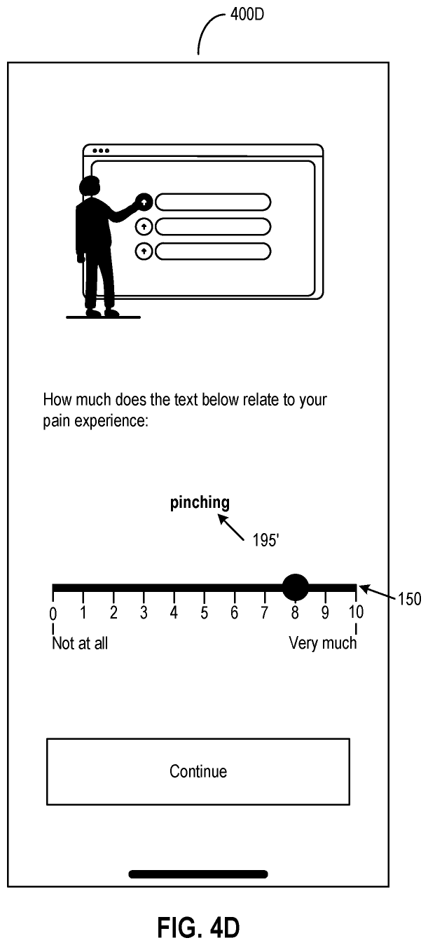

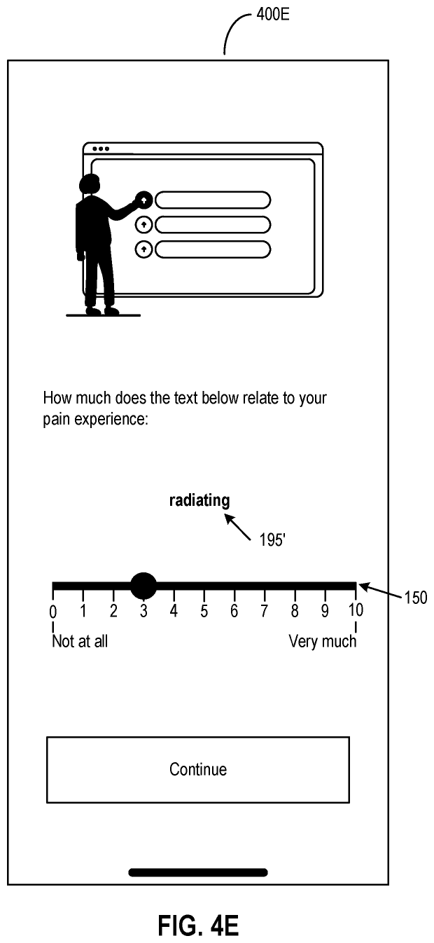

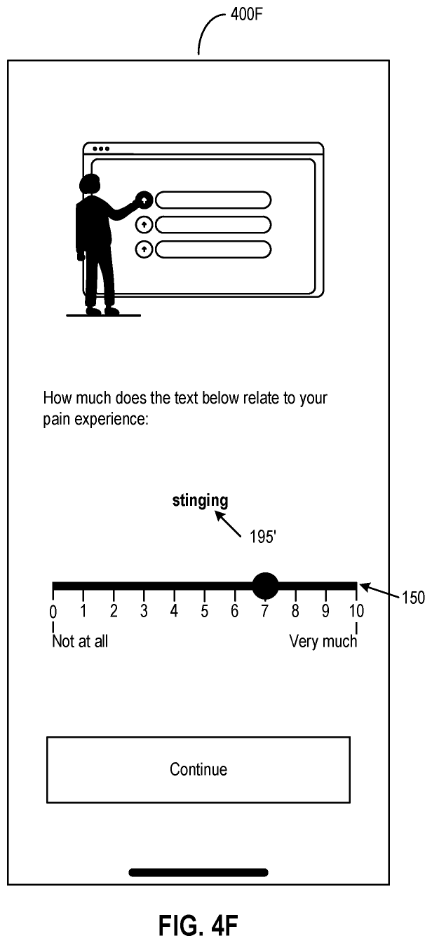

In conjunction with presentation of each stimulus 195′, the profile creator 130 may provide or present the initiation prompt 205 on the user interface 145 to receive one or more indications 220 for the presented stimulus 195′ from the user 210. The initiation prompt 205 may be presented subsequent to or at least partially concurrent with the presentation of the corresponding stimulus 195′. The initiation prompt 205 at this stage may identify or include a set of questions for the user 210 to indicate a degree of relevance of the presented stimulus 195′ to the condition to be addressed. Examples of the user interface 145 for the initiation prompt 205 may include text with the question about the degree or relevance that may be found in FIGS. 4A-F. For instance, the user interface 145 for the initiation prompt 205 may include text with the question “How much does the word relate to your pain?” for stimuli 195′ to address the condition of chronic pain on the part of the user 210. The user interface 145 for the initiation prompt 205 may include one or more UI elements 150 that can be used by the user 210 to input the degree of relevance to the chronic pain.

The profile creator 130 may retrieve, receive, or otherwise identify at least one indication 220 from the user 210. The indication 220 may be entered or inputted by the user 210 through the UI elements 150 of the user interface 145 for the initiation prompt 205 in response to presentation of the stimulus 195′. The indication 220 may identify or include a relevance value indicative of the degree of relevance of the presented stimulus 195′ to the condition of the user 210. The relevance value may be a numeric value (e.g., between 0 to 10, −1 to 1, −10 to 10, or 0 to 100) indicating the degree of relevance of the stimulus 195′ to the condition as identified by the user 210. For instance, in the user interface 145 for the initiation prompt 205, the user 210 may have selected the UI element 150 corresponding to the degree of the relevance of the stimulus 195′ to the condition to be addressed, upon presentation of the stimulus 195′. The profile creator 130 may parse the input received from the user 210 on the user interface 145 for the initiation prompt 205 to identify the corresponding relevance value. In some embodiments, the profile creator 130 may receive the indication 220 identifying the relevance value from a clinician evaluating the user 210 upon presentation of the stimulus 195′.

In some embodiments, the profile creator 130 may retrieve, identify, or otherwise receive a set of physiological measurements of the user 210 in response to the presentation of the stimulus 195′ to the user 210. The physiological measurements may be acquired from sensors on the user 210 subsequent to or partially concurrent with the presentation of the stimulus 195′. These sensors can include a heart rate sensor, a pulse oximetry sensor, a temperature sensor, motion sensor, and a body posture sensor, among others. The physiological measurements may include, for example, heart rate, heart rate variability, breathing rate, oxygen levels, temperature, skin conductance (sweat response), and posture, among others. Based on the received physiological measurements, the profile creator 130 may calculate, generate, or otherwise determine the relevance value for the stimulus 195′ to the condition. In some embodiments, the determination may be in accordance with a function between the physiological measurements with the degree of relevance. For example, the function may specify that the higher the heart rate as a result of the presentation of the stimulus 195′, the higher the degree of relevance of the stimulus 195′. Conversely, the function may specify that the lower the heart rate as the result of the presentation, the lower of the degree of relevance.

Based on the relevance value, the profile creator 130 may categorize or classify the presented stimulus 195′ as having a non-neutral reaction type (or not non-neutral reaction type). The stimulus 195′ presented to the user 210 may be one of the non-neutral stimuli 190 to determine whether the user 210 is particularly affected by the stimulus 195′ in relation to the condition or the neutral stimulus 190′ to confirm that the stimulus 195′ is neutral with respect to the condition of the user 210. To classify, the profile creator 130 may compare the relevance value with a threshold. The threshold may delineate a value for the relevance value at which the presented stimulus 195′ is classified as having a non-neutral reaction type or not. The non-neutral reaction type may be a reaction that has (1) idiosyncratically relevance to the user 210 and the condition to be addressed and (2) an emotional valence on the part of the user 210, among others. A neutral reaction type may be a reaction that lacks (1) idiosyncratic relevance to the user 210 or (2) emotional valence on the part of the user 210.

The profile creator 130 may determine or identify the threshold to compare against the relevance value indicated by the user 210. In some embodiments, the profile creator 130 may identify the threshold may be based on the condition of the user 210 to be addressed or the session to address the condition. The session may have one or more parameters defining various aspects of the session. The parameters may identify or include, for example, the condition of the user 210 to be addressed; types of the stimulus 195′ to be delivered; a type of task to be performed during the session; intensity for delivering the stimulus 195′ during the task; and length of the presentation of the stimulus 195′, among others. As a function of the parameters for the session, the profile creator 130 may determine the threshold. For instance, when the parameters specify that intensity of stimuli 195′ to be delivered is relatively high, the profile creator 130 may determine a higher threshold relative to when parameters specify that the intensity is to be low.

In some embodiments, the profile creator 130 may determine or identify the threshold based on another stimulus 195′ presented to the user 210. The other stimulus 195′ may have been previously presented by the profile creator 130 to the user 210. The determination of the threshold to compare against the relevance value for the subsequently presented stimulus 195′ may be based on a function of relevance values of one or more previously presented stimuli 195′, whether each previously presented stimulus 195′ is classified as having the non-neutral reaction type, and a number of previously presented stimuli 195′ classified as the non-neutral reaction type (or a not non-neutral reaction type), among others. For instance, the function may specify that if the number of previously presented stimuli 195′ classified as a non-neutral reaction type is greater than or equal to a threshold number, the threshold is to be increased. Conversely, the function may specify that if the number of previously presented stimuli 195′ classified as a not non-neutral reaction type is greater than or equal to another threshold number, the threshold is to be decreased.

From comparing the relevance value with the threshold, the profile creator 130 may determine whether the presented stimulus 195′ has the non-neutral reaction type to the condition to be addressed. If the relevance value is determined to satisfy (e.g., greater than or equal to) the threshold, the profile creator 130 may classify the stimulus 195′ as having the non-neutral reaction type. When the presented stimulus 195′ is classified as having the non-neutral reaction type, the stimulus 195′ may trigger, activate, or otherwise elicit a response particular to the user 210 (e.g., an idiosyncratic stressor) with respect to the condition to be addressed. In the case where the stimulus 195′ is one of the non-neutral stimuli 190, the profile creator 130 may determine that the stimulus 195′ is confirmed to have the non-neutral reaction type particular to the user 210. In some embodiments, the profile creator 130 may store and maintain the stimulus 195′ and the classification of the stimulus 195′ as having the non-neutral reaction type on the user device 110.

Conversely, if the relevance value is determined to not satisfy (e.g., is less than) the threshold, the profile creator 130 may classify the stimulus 195′ as not having the non-neutral reaction type. When the presented stimulus 195′ is classified as not having the non-neutral reaction type, the stimulus 195′ may not trigger or elicit the response particular to the user 210 with respect to the condition to be addressed. In the case where the stimulus 195′ is one of the neutral stimuli 190′, the profile creator 130 may determine that the stimulus 195′ is confirmed to have the neutral reaction type. In some embodiments, the profile creator 130 may store and maintain the stimulus 195′ and the classification of the stimulus 195′ as not having the non-neutral reaction type on the user device 110. In some embodiments, the profile creator 130 may discard or remove the stimulus 195′ from the initial set stored on the user device 110 upon classifying the presented stimulus 195′ as not having the non-neutral stimulus reaction type.

In some embodiments, when the presented stimulus 195′ is classified as having the non-neutral reaction type, the profile creator 130 may identify or select at least one neutral stimulus 190′ to associate with the stimulus 195′. The neutral stimulus 190′ may be from the initial set of stimuli 195′ provided by the session configuration system 105 or may be retrieved from the database 175 maintaining the set of neutral stimuli 190′ for addressing the condition. To select, the profile creator 130 may identify one or more characteristics of the presented stimulus 195′ and each neutral stimulus 190′. The characteristics may identify or include length (e.g., duration in presentation), intensity (e.g., audio volume), color values (e.g., red-green-blue (RGB) values for image or video), topic category (e.g., from knowledge graph), and frequency in use (e.g., language corpus database), among others.

The profile creator 130 may compare the characteristics of each neutral stimulus 190′ with the characteristics of the presented stimulus 195′. Using the comparison of the characteristics, the profile creator 130 may calculate or determine a similarity metric. The similarity metric may indicate a degree of similarity in presentation and topic between the presented stimulus 195′ and the neutral stimulus 190′. With the determination, the profile creator 130 may compare the similarity metric with a threshold. The threshold may identify a value for the similarity metric at which the presented stimulus 195′ and the neutral stimulus 190′ are to be paired or associated. When the similarity metric satisfies (e.g., is greater than or equal to) the threshold, the profile creator 130 may determine that the presented stimulus 195′ and the neutral stimulus 190′ are to be associated. The profile creator 130 may also produce, output, or otherwise generate a set (or a pair) identifying or include the presented stimulus 195′ and the neutral stimulus 190′. In some embodiments, multiple neutral stimuli 190′ may be associated by the profile creator 130 with the stimulus 195′ to form a set of stimuli. On the other hand, when the similarity metric does not satisfy (e.g., is less than) the threshold, the profile creator 130 may determine that the presented stimulus 195′ and the neutral stimulus 190′ are not to be associated. The profile creator 130 may continue to traverse through the neutral stimuli 190′ to find the neutral stimulus 190′ to associate with the presented stimulus 195′.

With the determination of the classifications, the profile creator 130 may produce, output, or otherwise generate an association between the user 210 and one or more presented stimuli 195′ classified as having the non-neutral reaction type. The association may be generated in accordance with one or more data structures, such as a linked list, a tree, a table, an array, a graph, a heap, or a hash table, among others. The association may identify or include an identifier referencing the user 210 (e.g., a unique user identifier (UUID), account identifier, application identifier for the application 120, and network address for the user device 110, among others) and identifiers corresponding to the stimuli 195′ classified as the non-neutral reaction type. In some embodiments, the profile creator 130 may generate the association between the user 210 and the set of the stimulus 195′ classified as having the non-neutral reaction type and the neutral stimulus 190′. The set for the association may include the identifiers corresponding to the stimulus 195′ and the associated neutral stimulus 190′. In some embodiments, the profile creator 130 may store and maintain the association for the user 210 on the user device 110. In some embodiments, the profile creator 130 may send, provide, or transmit the association to the session configuration system 105 for storage and maintenance on the database 175.

With the generation, the profile creator 130 may produce, output, or otherwise generate the user profile 180 for the user 210 to include the association. The user profile 180 may include or identify the association between the user 210 and the one or more stimuli 195′ classified as having the non-neutral reaction type. In some embodiments, the user profile 180 may include or identify the association between the user 210 and the set of the stimulus 195′ and the neutral stimulus 190′. In addition, the user profile 180 may identify or include the identifier for the user 210, the condition to be addressed, the session to be used to address the condition, number of stimuli 195′ classified as having the non-neutral reaction type, number of stimuli 195′ classified as not having the non-neutral reaction type, and the type of stimuli for the session, among others. Upon generation, the profile creator 130 may provide, send, or otherwise transmit the user profile 180 (or the association itself) to the session configuration system 105.

The profile assessor 155 may retrieve, identify, or otherwise receive the user profile 180 (or the association itself) from the application 120 executing on the user device 110. Upon receipt, the profile assessor 155 may parse the user profile 180 to extract or identify the association between the user 210 and the stimuli 195′ classified as having the non-neutral reaction type to the user 210. In some embodiments, from parsing, the profile assessor 155 may identify the association between the user 210 and the set of the stimulus 195′ and the neutral stimulus 190′. In conjunction, the profile assessor 155 may store and maintain the user profile 180 (or the association parsed therefrom) in the database 175.

The package creator 160 executing on the session configuration system 105 may identify or select at least one configuration file 185 from the set of configuration files 185 stored and maintained in the database 175, using the user profile 180 (or the association). The selected configuration file 185 may include instructions for performing the session by providing the stimuli 195′ to address the condition of the user 210. For instance, when the user profile 180 specifies that emotion regulation is the condition of the user 210 to be addressed, the package creator 160 may select the configuration file 185 corresponding to a session to address emotion regulation.

The configuration file 185 may include or identify task logic 225 and a set of stimulus identifiers 230A-N (hereinafter generally referred to as stimulus identifiers 230). The task logic 225 may define, specify, or otherwise include instructions for running the session on the application 120. Each stimulus identifier 230 may correspond or reference the stimulus 195′ to be delivered to the user 210 via the application 120. The set of stimulus identifiers 230 may correspond to the stimuli 195′ classified as having the non-neutral reaction type. In some embodiments, depending on the type of session to be carried out, the set of stimulus identifiers 230 may include the stimuli 195′ classified as having the non-neutral reaction type and corresponding, associated neutral stimuli 190′. In some embodiments, the set of stimulus identifiers 230 may include neutral stimuli 190′ pre-designated for the session, along with the stimuli 195′ classified as having the non-neutral reaction type.

The session may include one or more tasks that the user 210 is to carry out via the application 120 to address the behavioral or psychological condition of the user 210. The tasks may be to activate cortical higher order cognitive systems, as the user 210 is presented with personally relevant and emotional trigger information via the stimuli 195′ as identified in the stimulus identifiers 230. The simultaneous stimulation of cortical systems and emotion processes may improve regulation abilities, lead to more adaptive and improved emotional reactions with the potential to improve symptoms, disease progression, risk of relapse or improve response on the part of the user 210 to other therapies. The tasks to be carried may include, for example, a personal trigger memory task, an implicit association task (IAT), attention bias modification training (ABMT), and go/no-go training, among others. Details of the performance of the task by the user 210 is detailed hereinbelow in conjunction with FIG. 2B.

With the identification, the package creator 160 may write, produce, or otherwise generate at least one session package 235 in accordance with the selected configuration file 185. The session package 235 may include machine-readable code to be executed by the application 120 to run the session as defined by the configuration file 185. The session package 235 may, for example, be in an intermediate-level or lower-level language compiled from the configuration file 185 and to be read by the user device 110 to carry out the instructions. The instructions included in the session package 235 may include the following functions: to modify the UI elements 150 of the user interface 145, to receive responses from the user 210, and to present the stimuli 195 corresponding to the stimulus identifiers 230. In some embodiments, the package creator 160 may include the configuration file 185 in the session package 235 for the application 120 to compile and execute. In some embodiments, the package creator 160 may include the stimuli 195 corresponding to the stimulus identifiers 230 defined by the configuration file 185 in the session package 235. Once generated, the package creator 160 may send, transmit, or otherwise provide the session package 235 to the application 120 on the user device 110.

Referring now to FIG. 2B, depicted is a block diagram for a process 250 to execute sessions to deliver stimuli to users in the system 100. The process 250 may include or correspond to operations performed in the system 100 to provide a session by presenting stimuli 195 to the user 210. Under the process 250, the session manager 135 of the application 120 executing on the user device 110 may initiate, load, or otherwise execute the session package 235 received from the session configuration system 105. In some embodiments, the session manager 135 may parse and compile the instructions in a similar manner as described above as performed by the package creator 160. In parsing and compiling, the session manager 135 may present or provide instructions for presentation of the stimulus 195 in the session to address the condition of the user 210. Upon loading, the session manager 135 may perform, execute, or otherwise carry out the session to address the condition of the user 210.

For the session, the session manager 135 may retrieve, receive, or otherwise identify the set of stimuli 195″A-N (hereinafter generally referred to as stimuli 195″) to be presented to the user 210. The stimuli 195″ may include at least one of the stimuli 195′ classified as having the non-neutral reaction type on the part of the user 210. In some embodiments, the set of stimuli 195″ may include at least one neutral stimulus 190′ to be presented with the non-neutral stimuli 190. In some embodiments, the stimuli 195″ may be identified or included in the session package 235. The session manager 135 may parse the session package 235 to identify or extract the identifiers for the stimuli 195″ or the files corresponding to the stimuli 195″.

In some embodiments, the session manager 135 may access the storage on the user device 110 to retrieve, receive, or identify the association or the set of stimuli 195′ stored and maintained on the user device 110. In some embodiments, the set of stimuli 195″ may correspond to the stimuli 195′ maintained on the user device 110 from the process 200. From the association, the session manager 135 may identify the set of stimuli 195′ to use as the stimuli 195″ to be provided for the session. In some embodiments, the session manager 135 may identify each set of stimuli from the association, including the stimulus 195′ classified as having the non-neutral reaction type and the neutral stimulus 190′. With the identification, the session manager 135 may use the identified stimuli 195′ as the stimuli 195″ for presentation in the session.

In some embodiments, the session manager 135 may send, provide, or otherwise transmit a request 255 for stimuli to the session configuration system 105. The request may be generated and sent when the files for the stimuli 195″ are not provided with the session package 235, and thus not loaded in storage on the user device 110. The request 255 for stimuli may include identifiers for the set of stimuli 195″ as specified in the session package 235 to be presented to the user 210 during the session.

The stimuli deliverer 165 executing on the session configuration system 105 may retrieve, identify, or otherwise receive the request 255 for stimuli from the application 120 running on the user device 110. Upon receipt, the stimuli deliverer 165 may parse the request 255 to identify or extract the identifiers for the set of stimuli 195″. For each identifier, the stimuli deliverer 165 may access the database 175 to find, retrieve, or otherwise identify the requested stimulus 195″. For example, the stimuli deliverer 165 may find the files for the requested stimuli 195″ corresponding to the file names as identified in the request 255. The requested stimuli 195″ may include the non-neutral stimuli 190 or the neutral stimuli 190′ as identified in the request 255. With the identification, the stimuli deliverer 165 may provide, send, or otherwise transmit the set of stimuli 195″ as requested and identified from the database 175 to the application 120 running on the user device 110. The session manager 135 may in turn retrieve, identify, or otherwise receive the set of stimuli 195″ from the session configuration system 105.

In carrying out the session, the session manager 135 may provide or present the stimuli 195″ to the user 210 in accordance with the session as defined by the session package 235. One or more stimuli 195″ may be provided at a time as specified by the session package 235 to run the session. For example, the session manager 135 may present a first stimulus 195″A (e.g., one of the stimuli 195′ classified as having the non-neutral reaction type) and a second stimulus 195″B (e.g., one of the neutral stimuli 190′) together on the user interface 145 of the application 120. To provide the stimulus 195″ to the user 210, the session manager 135 may display, play, or otherwise present the stimulus 195″ via an input/output (I/O) device of the user device 110. For example, for a textual or image content, the session manager 135 may render or display the stimulus 195″ via the user interface 145 as at least one of the UI elements 150. For audio content, the session manager 135 may play the stimulus 195″ via the speakers of the user device 110. For video or multimedia content, the session manager 135 may present the visual portion of the stimulus 195″ via the user interface 145 of the application 110 and play the audio portion of the stimulus 195″ via the speakers. The stimuli 195″ presented in at least partial concurrence (e.g., with the other stimuli) may be referred to as a block within the session provided to the user 210 to address the condition.

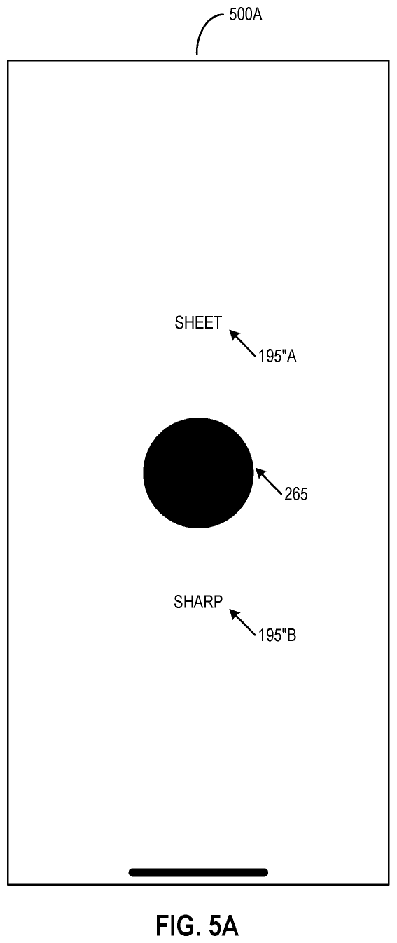

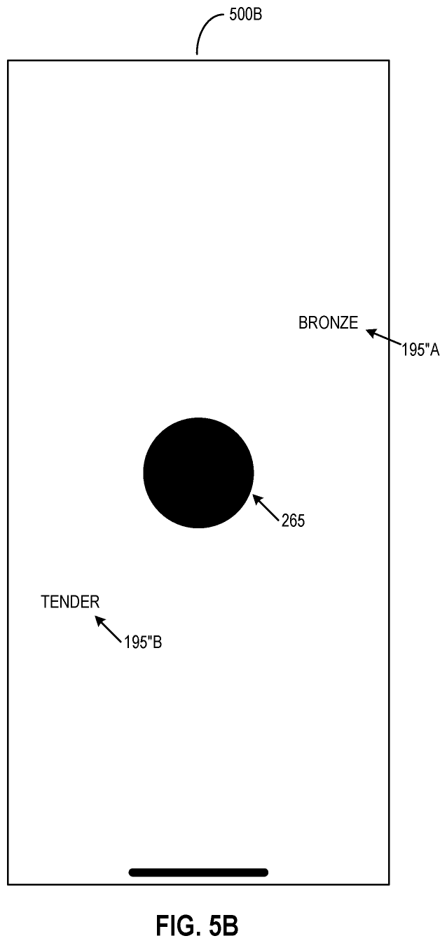

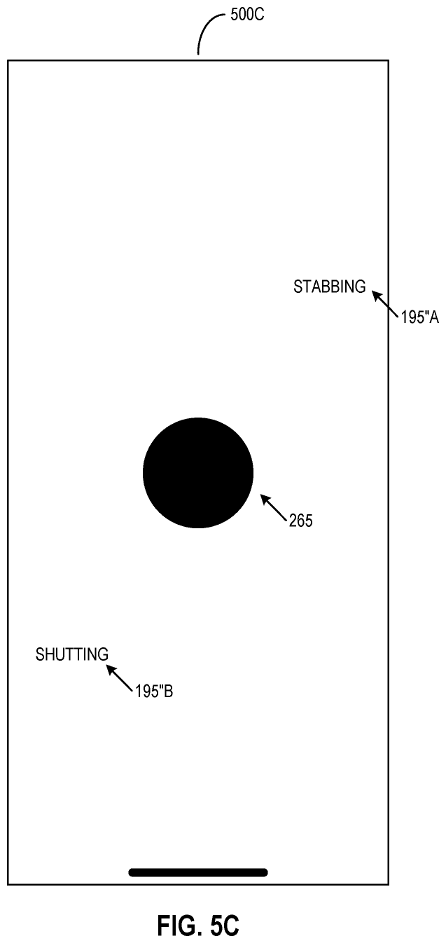

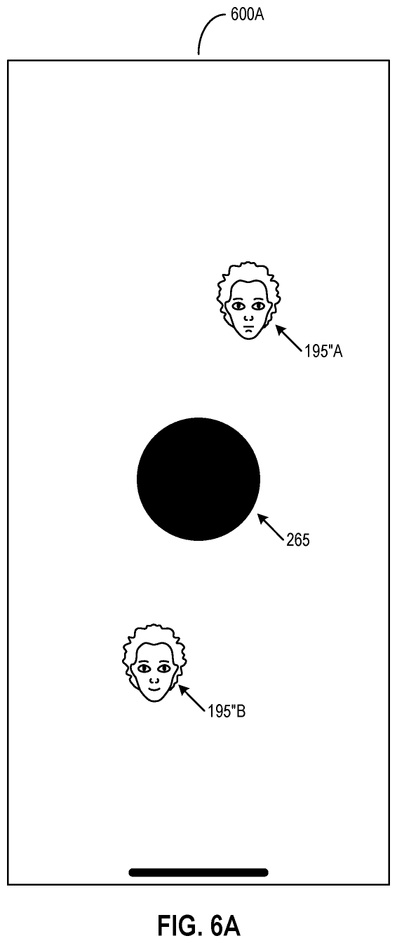

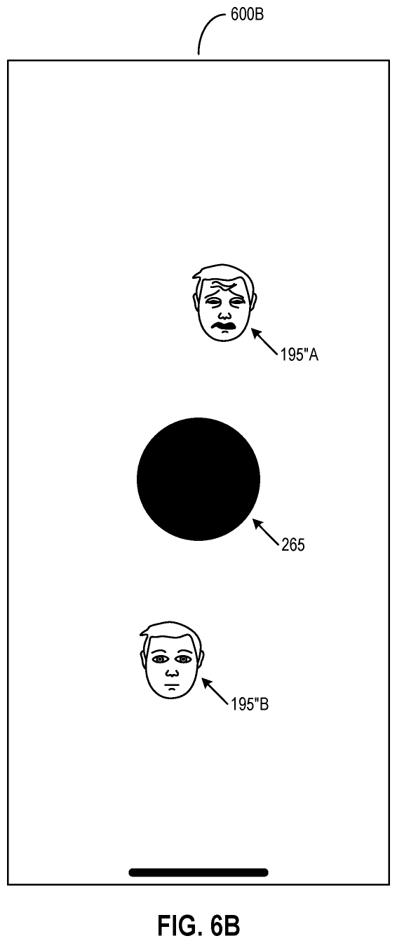

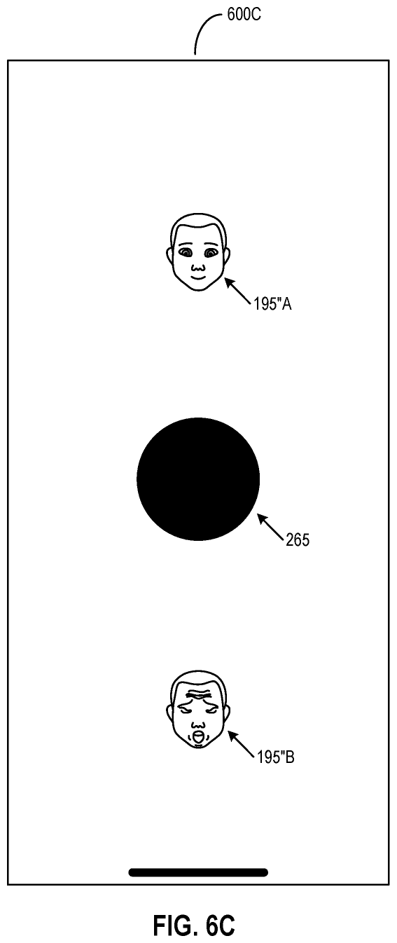

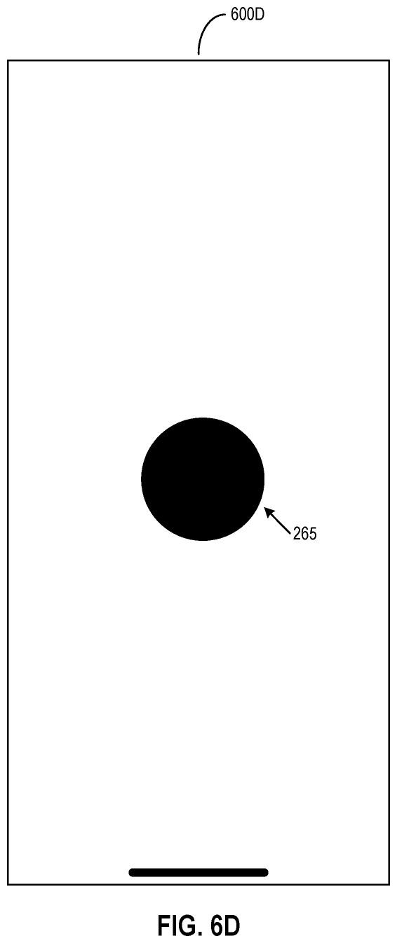

In conjunction with the presentation of the one or more stimuli 195″, the session manager 135 may provide or present at least one cue 265. The presentation of the cue 265 may be subsequent to or at least partially concurrent with the presentation of the one or more stimuli 195″. The cue 265 may be configured to guide, lead, or otherwise direct the focus of the user 210 toward or away from at least one of the presented stimuli 195″. For example, the cue 265 may be positioned closer to the neutral stimulus 190′ presented within the user interface 145 than to the stimulus 195″ classified as having the non-neutral reaction type. In this manner, the user 210 may disassociate the stimuli 195″ from the negative effects of the behavioral or psychological condition in the brain of the user 210. Examples of the presentation of the stimuli 195″ with the cue 265 for the session are described herein in conjunction with FIGS. 5A-C (e.g., using word stimuli) and FIGS. 6A-D (e.g., using facial expression stimuli).

The session manager 135 may configure the presentation of the stimuli 195″ and the cue 265 in accordance with the specification of the session package 235. The configuration (e.g., size, temporal or graphical placement, intensity, and length) of the cue 265 may depend on the task to be performed in the session. In some embodiments, the session manager 135 may present the cue 265 at least partially concurrent to the presentation of the stimuli 195″ to increase the likelihood of selection of the stimulus 195″ with a target reaction type. The target reaction type may be defined in the session package 235 and may be dependent on the task to be performed by the user 210 to address the user's condition. For certain sessions, the target reaction type for the stimulus 195″ may be the neutral reaction type. In accordance with the definition of the session package 235, the session manager 135 may present the cue 265 proximate to the neutral stimulus 190′ to increase the likelihood that the user 210 reacts to the neutral stimulus 190′ (e.g., IAT). In some embodiments, the session manager 135 may present the cue 265 proximate to the neutral stimulus 190′ to decrease the likelihood of reaction to the non-neutral stimulus 190 (e.g., go/no-go and ABMT).

Conversely, for certain sessions, the target reaction type for the stimulus 195″ may be the non-neutral reaction type. In accordance with the definition of the session package 235, the session manager 135 may present the cue 265 proximate to the neutral stimulus 190′ to decrease the likelihood that the user 210 reacts to the neutral stimulus 190′. In some embodiments, the session manager 135 may present the cue 265 to increase the likelihood of reaction to the non-neutral stimulus 190. Additional factors in the configuration of the presentation of the stimuli 195″ and the cue 265 are detailed herein below.

For the IAT task (also referred to herein as self-processing task), the problem on the part of the user 210 to be addressed may include repeated association of the user 210 themselves with chronic conditions. The chronic pain can lead to non-adaptive association of the self as ill or to feelings of guilt, shame, or self-criticism (e.g., failure for being ill and shame of not functioning), among others. In the IAT task, the user 210 may be prompted via the user interface 215 to associate the neutral stimulus 190′ with the user 210 themselves and disassociate personally relevant negative non-neutral stimulus 190 as quick as possible (associate those stimuli with others). Through repeat pairing of non-negative, neutral stimuli 190′ with the self, and negative relevant self-appraisals with ‘other’ or non-self, maladaptive self-processes may be rectified.

The stimuli 195″ presented during the IAT task to the user 210 may cause the user 210 to disassociate the non-neutral stimuli 190 from the self and associate the neutral stimuli 190′ with the self In this manner, under the IAT task, the stimuli 195″ may be presented to increase the likelihood that the user 210 select the neutral stimuli 190′ to associate the self with the neutral stimuli 190′ and disassociate the self from non-neutral (negative, self-critical) stimuli 190. Conversely, the stimuli 195″ may be presented to decrease the likelihood that the user 210 select the non-neutral stimuli 190 to disassociate the self with the non-neutral stimuli 190 and associate the self with the neutral stimuli 190′. In some embodiments, the stimuli 195″ may be presented without the cue 265. The task may activate default mode and emotion processing brain regions within the user 210: emotion processing systems (e.g., amygdala) for self-related negative stimuli (e.g., self-criticism, shame, and illness), and default mode, cortical midline structures of the brain, including medial prefrontal cortex (PFC), posterior cingulate cortex, and anterior precuneus (self-processing structures), among others.

For the personal trigger memory task (also referred to herein as emotion regulation), the problem on the part of the user 210 to be addressed may include decreased emotion regulation abilities with stimuli 195 related to indications. The issues may include PTSD, depression, addiction, chronic pain, reactivity to stressors related to the indication, among others. By being able to select personal stressors or triggers via the stimuli 195′ classified as having the non-neutral reaction type for the personal trigger memory (with the aid of the cue 265), the training can strengthen and improve emotion regulation and cognitive control across indications. In addition, the stimuli 195 may also aid in controlling and regulating personal trigger memory. To train, the user 210 may perform n-back tests with a certain percentage of emotional or triggering stimuli (e.g., the non-neutral stimuli 190) and a certain percentage of neutral stimuli 190′. In some embodiments, a set of stimuli 195 used for the n-back tests of the personal trigger memory task may include a certain percentage of neutral stimuli 190′ and a remaining percentage of non-neutral stimuli 190. The percentage of emotional stimuli per round or training session can be increased and the time allotted for the response may be decreased, to increase difficulty or challenge to the emotional system of the user 210. The task may strengthen emotion regulation abilities in the user 210. The targeted brain regions may include prefrontal areas activated through the n-back task and emotion processing regions activated through the non-neutral stimuli.

For the ABMT task, the problem to be addressed may include increased attention capture and hyper alertness to indicate related stressors or emotional stimuli (e.g., the non-neutral stimuli 190), such as hypersensitivity to words or images related to pain in users 210 chronic pain or being hyper-alert to fear-related stimuli in anxiety. To train, the user 210 may be retrained to direct attention away from the attention capturing stimuli (e.g., the non-neutral stimuli 190), when prompted to react to the cue 265 appearing behind the visual, neutral stimuli 190′. This may have the effect of decreasing the likelihood of selecting the non-neutral stimuli 190. This task may decrease attention capture and increase attentional flexibility when dealing with emotional stimuli. The targeted brain regions within the user 210 may include emotion processing (stimuli) and attention network, among others.

For the go/no-go task (sometimes herein referred to as inhibition), the problem to be addressed may include that emotional stimuli may trigger maladaptive responses, such as smoking as a reaction to stress triggers. To train, the user 210 may be prompted (e.g., using the cue 265) to ignore and not to react to the non-neutral stimuli 190 (also referred to herein as a no-go stimulus) and instead to focus and react to the relevant neutral stimuli 190′ (also referred to herein as a go-stimulus). This may have the effect of decreasing the likelihood of the user 210 selecting the non-neutral stimuli 190. The targeted brain regions may include emotional response system (e.g., amygdala) and emotional or impulse control system (dorsomedial prefrontal cortex and rostral anterior cingulate cortex).

In addition, the session manager 135 may present or provide at least one session prompt 260 to the user 210 of the application 120 via the user interface 145 to receive one or more responses 270 from the user 210. The presentation of the session prompt 260 may be subsequent to or at least partially concurrent with the presentation of the one or more stimuli 195″. The responses 270 may be entered or inputted by the user 210 through the one or more UI elements 150 of the user interface 145 for the session prompt 260. The response 270 may indicate or identify the selection by the user of the stimulus 195″. The selected stimulus 195″ may be one of set of stimuli 195″, including the stimulus 195′ classified as having the non-neutral reaction type or the neutral stimulus 190′.

The session manager 135 may monitor for entry or inputting of the response 270 by the user 210 via the session prompt 260, in conjunction with the presentation of the one or more stimuli 195″. Upon detection of the entry, the session manager 135 may parse the response 270 to identify the stimulus 195″ selected by the user 210. In some embodiments, the session manager 135 may compare the reaction type of the selected stimulus 195″ with the target reaction type for the session as specified by the session package 235. If the reaction type differs from the target, the session manager 135 may determine that the selected stimulus 195″ is incorrect. Conversely, the reaction type is the same as the target, the session manager 135 may determine that the selected stimulus 195″ is correct. The session manager 135 may store a record of the determination with the target reaction type for the presentation of the stimuli 195″.

In some embodiments, the session manager 135 may maintain a timer to keep track of time elapsed between the presentation of the one or more stimuli 195″ (or the cue 265) and the receipt of the response 270 from the user 210. Using the timer, the session manager 135 may identify, measure, or otherwise determine the reaction time of the user 210 in response to the presentation of the stimuli 195″. With the entry of the response 270, the session manager 135 may identify the set of stimuli 195″ to be presented from the session package 235 and repeat the process again until all the stimuli 195″ have been presented. The session manager 135 may store a record of the reaction time of the user 210 for the presentation of the stimuli 195″.

The progress evaluator 140 of the application 120 executing on the user device 110 may produce, output, or otherwise generate session data 275 based on the responses 270 from the user 210 in response to the presentation of the stimuli 195″. The session data 275 may identify or indicate the performance of the user 210 in carrying out the tasks in accordance with the session. The session data 275 may identify or include the identifier for the user 210, the identifiers for the set of stimuli 195″ presented to the user, number of correct or incorrect responses by the user 210, the response 270 identifying the selection of the stimuli 195″, and the reaction time, among others. The session data 275 may include records of the responses 270 arranged by blocks for the session. Upon generation, the progress evaluator 140 may transmit, send, or provide the session data 275 to the session configuration system 105.

The record tracker 170 executing on the session configuration system 105 may retrieve, identify, or otherwise receive the session data 275 from the application 120 executing on the user device 110. Upon receipt, the record tracker 170 may parse the session data 275 to extract or identify the contents therein, such as the identifier for the user 210, identifiers for the presented stimuli 195″, number of correct or incorrect responses by the user 210, the response 270 identifying the selection of the stimuli 195″, and the reaction time, among others. The record tracker 170 may store and maintain the records of the responses from the session data 275 onto the database 175. In some embodiments, the record tracker 170 may add the session data 275 to the user profile 180 for the user 210 in the database 175.

Using the session data 275, the progress evaluator 140 (or the record tracker 170) may update, set, or otherwise modify the parameters of the session for the next delivery to the user 210. In some embodiments, based on the responses 270, the progress evaluator 140 may calculate, generate, or determine a difficulty measure of the user 210 in performing the tasks as specified by the session. The difficulty measure may indicate how difficult the tasks were to the user 210 in terms of correctness with selecting the stimuli 195″ of the target reaction type or reaction type to the presentation of the stimuli 195″. In general, the greater the incorrect responses and higher the reaction time, the higher the difficulty measure may be. Conversely, the lower the incorrect responses and the lower the reaction time, the lower the difficulty measure may be.

The progress evaluator 140 may compare the difficulty measure with a baseline value. The baseline may define a value at which to adjust the parameters of the session, given the ease or difficulty of the session. If the difficulty measure is lower than the baseline, the progress evaluator 140 may determine that the session was easy for the user 210 and may increase the difficulty for the session in the next instance by adjusting the parameters. For instance, the progress evaluator 140 may increase the threshold for the relevance value in determining whether the stimulus 195′ is non-neutral to the condition of the user 210. Conversely, if the difficulty measure is higher than the baseline, the progress evaluator 140 may determine that the session was difficult for the user 210 and may decrease the difficulty for the session in the next instance by adjusting the parameters. For example, the progress evaluator 140 may decrease the threshold for the relevance value in determining whether the stimulus 195′ is non-neutral to the condition of the user 210. The progress evaluator 140 may store and maintain the adjusted parameters for the session on the user device 110.

In some embodiments, the progress evaluator 140 (or the record tracker 170) may also perform or carry out an evaluation of the user 210 in addressing the behavioral or psychological condition. In some embodiments, the progress evaluator 140 may receive or identify a first evaluation set for the user 210 prior to the session including the presentation of the stimuli 195″. In conjunction, the progress evaluator 140 may receive or identify a second evaluation set for the user 210 subsequent to the session. Each evaluation set may identify measures (e.g., taken by a clinician) relevant to the behavioral or psychological condition of the user 210 to be treated. The measures in the evaluation set may be acquired or identified separate to the session and the stimuli 195″ presented to the user 210.

With the identification, the progress evaluator 140 may generate or determine a progression for the user 210 in addressing the condition based on the evaluation sets. To determine the progression, the progress evaluator 140 may compare the measures in the evaluation set prior to the session with the measures in the evaluation set subsequent to the session. If the measures indicate an improvement (e.g., indication of less pain or lower incidence of smoking), the progress evaluator 140 may determine a positive progression for the user 210. On the other hand, if the measures indicate a degradation (e.g., indication of more pain or higher incidence of smoking), the progress evaluator 140 may determine a negative progression for the user 210. Upon determination, the progress evaluator 140 may store and maintain the progression on the user device 110 or provide the progression for storage in the database 175 with the user profile 180.

In this manner, by using feedback from the user 210, the application 120 running on the user device 110 may identify stimuli 195 with non-neutral reaction types in an objective manner. The identified stimuli 195 may further be particular to the idiosyncratic stressors of the user 210 to assist in treating the behavioral or psychological condition. Using the identified stimuli 195, the session configuration system 105 may generate a customized sessions unique to the particularities of the user 210, with stimuli 195 targeted at regions of the brain correlated with the condition. Moreover, the presentation of the stimuli 195″ via the application 120 and the recordation of the responses 270 may provide for a centralized manner of carrying out of sessions to provide stimuli 195 to the user 210, without reliance on tedious, manual recordation. These aspects improve the overall functioning of the computer in providing targeted neuro-stimulation and enhance the quality of human-computer interactions (HCI) between the user 210 and the application 120 running on the user device 110.

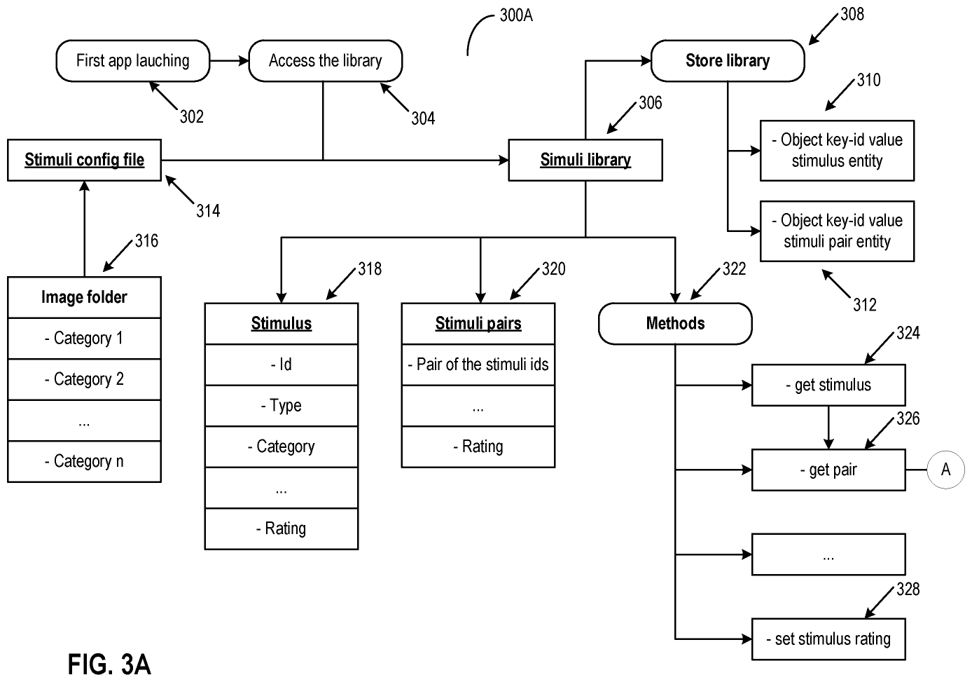

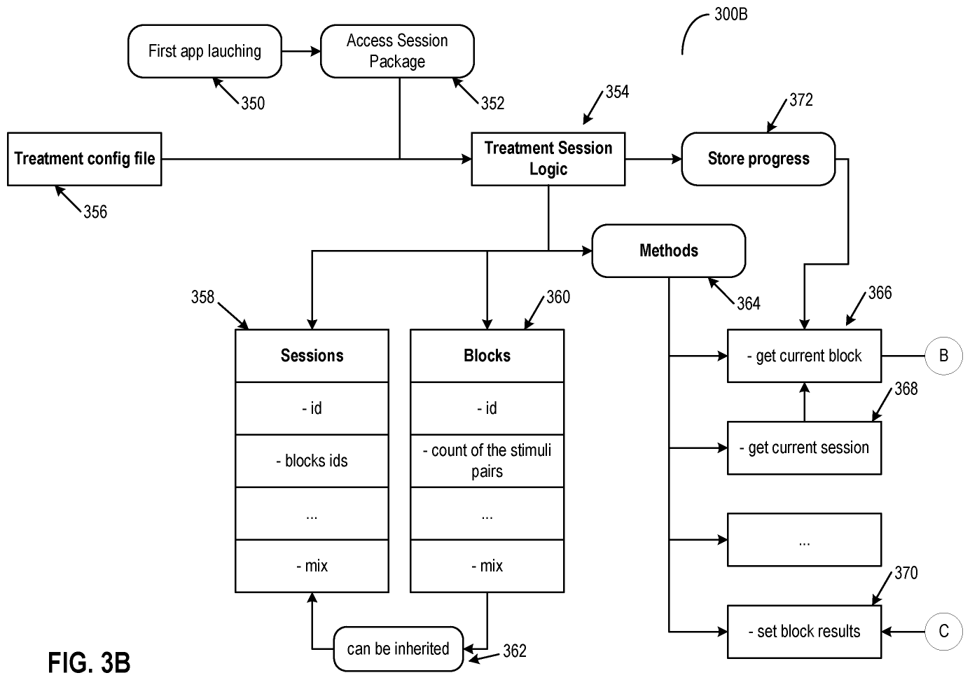

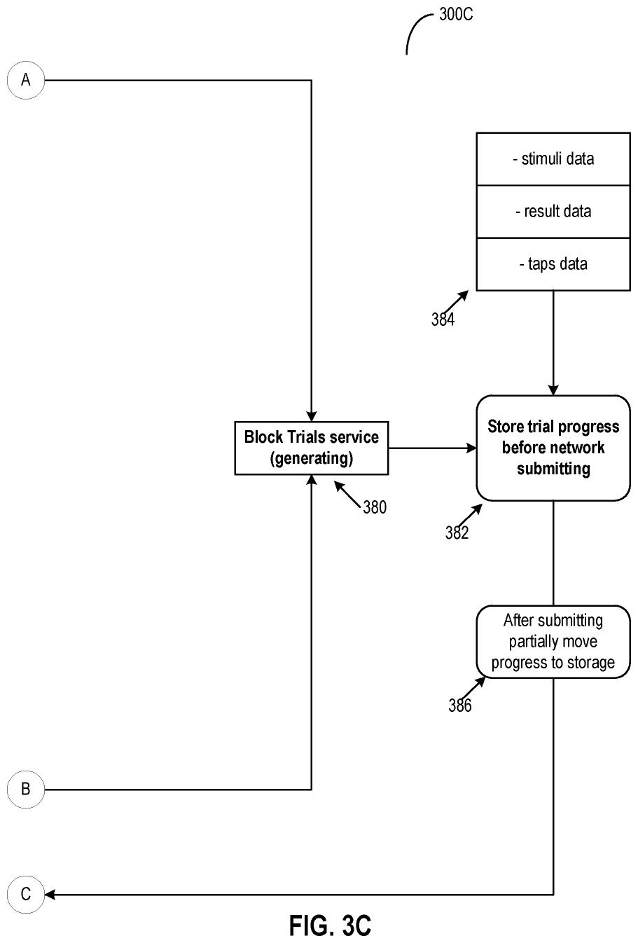

Referring now to FIGS. 3A-C, depicted are block diagrams of a system architecture for adaptively selecting stimuli to provide sessions. Focusing on FIG. 3A, depicted is architecture 300A for associating stimuli in the system. As shown, a computing system (e.g., the user device 110 or the session configuration system 105) may first launch the application (302) and access a library of stimuli (304). The stimuli library (306) may have a store library (308) with object-key identifier values for stimuli (310) and object-key identifier values for stimuli sets (or pairs) (312). The library may also have a stimuli configuration file (314) including an image folder of various categories (316). The library may also have the individual stimuli (318) indexed by identifiers, types, categories, and ratings (e.g., relevance values), among others, and individual sets (320) indexed by identifier for the set and rating, among others. The computing system may carry out various methods (322) to retrieve stimuli (324), retrieve sets of stimuli (326), and set stimuli ratings (328).

In addition, focusing on FIG. 3B, depicted is an architecture 300B for setting up sessions to provide stimuli in the system. The computing system may first launch the application (350) and may access the session package (352). The session package may have treatment session logic (354). The session logic may be based on a treatment configuration file (356). The session logic may include sessions (358) as defined by stimuli identifiers and block identifiers, among others. The session logic may also include blocks (360) which can inherit (362) sessions, and may include stimuli identifiers and set identifiers, among others. The computing system may carry out various methods (364) to load the current block of the session (366), to retrieve the current session (368), and the set block results (370). In performing the sessions, the computing system may store progress of the user (372). Turning to FIG. 3C, depicted is an architecture 300C for running sessions in the system. The computing system may carry out the block trials service (380). The computing system may store trial progress (382) identifying various data (384), such as stimuli data, result data, and interaction data, among others. The computing system may move progress to storage (386).

Referring now to FIGS. 4A-F, each depict a screenshot 400A-F of a user interface (e.g., the user interface 145) with a prompt (e.g., the initiation prompt 205) regarding a degree of relevance of a presented stimulus to a user (e.g., the user 210) with a condition to be addressed. In each screenshot 400A-F, the initiation prompt 205 may include the stimulus 195′ in the form of a word (e.g., “disable” in 400A, “endless” in 400B, “miserable” in 400C, “pinching” in 400D, “radiating” in 400E, and “stinging” in 400F) to be classified as having the non-neutral reaction type or not. The initiation prompt 205 may include at least one UI element 150 (e.g., the slider as indicated) for the user 210 to enter the degree of relevance.

Referring now to FIGS. 5A-C, each depict a screenshot 500A-C of a user interface (e.g., the user interface 145) with a prompt (e.g., the session prompt 260) regarding a degree of relevance of a presented stimulus (e.g., the stimulus 195″) to a user (e.g., the user 210) with a condition to be addressed. In each screenshot 500A-C, the session prompt 260 may provide a set of stimuli, such as the first stimulus 195″A and the second stimulus 195″B, both in the form of words (e.g., “sheet” and “sharp” in 500A, “tender” and “bronze” in 500B, and “shutting” and “stabbing” in 500C). At least one of the stimuli 195″A or 195″B may be a non-neutral stimulus 190, and the other stimuli 195″A or 195″B may be a neutral stimulus 190′. The session prompt 260 may also provide the cue 265 generally toward the middle of the user interface 145, and set the positioning of the stimuli 195″A or 195″B to direct the user 210 to the stimulus 195″A or 195″B with the target reaction type (e.g., neutral reaction type).

Referring now to FIGS. 6A-D, each depict a screenshot 600A-D of a user interface (e.g., the user interface 145) with a prompt (e.g., the session prompt 260) regarding a degree of relevance of a presented stimulus (e.g., the stimulus 195″) to a user (e.g., the user 210) with a condition to be addressed. In each screenshot 600A-C, the session prompt 260 may provide a set of stimuli, such as the first stimulus 195″A and the second stimulus 195″B, both in the facial expressions (e.g., with happy, angry, sad, fear, or disgusted emotions as part of a personal trigger memory task). In some embodiments, the stimuli 195″ may include other types of stimuli 195, without or without the cue 265, to run a personal emotion memory task. At least one of the stimuli 195″A or 195″B may be a non-neutral stimulus 190, and the other stimuli 195″A or 195″B may be a neutral stimulus 190′. The session prompt 260 may also provide the cue 265 generally toward the middle of the user interface 145 and set the positioning of the stimuli 195″A or 195″B to direct the user 210 to the stimulus 195″A or 195″B with the target reaction type (e.g., neutral reaction type). In screenshot 600D, the session prompt 260 may provide the cue 265 without the stimuli prior to the presentation of the stimuli, to keep the attention of the user 210.

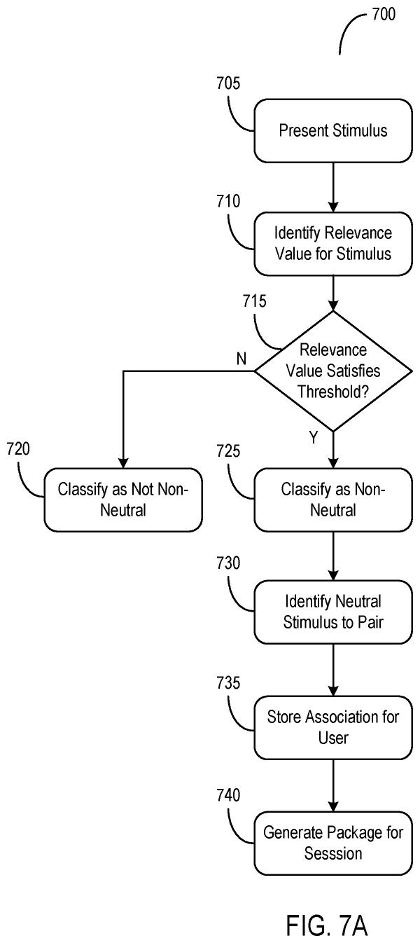

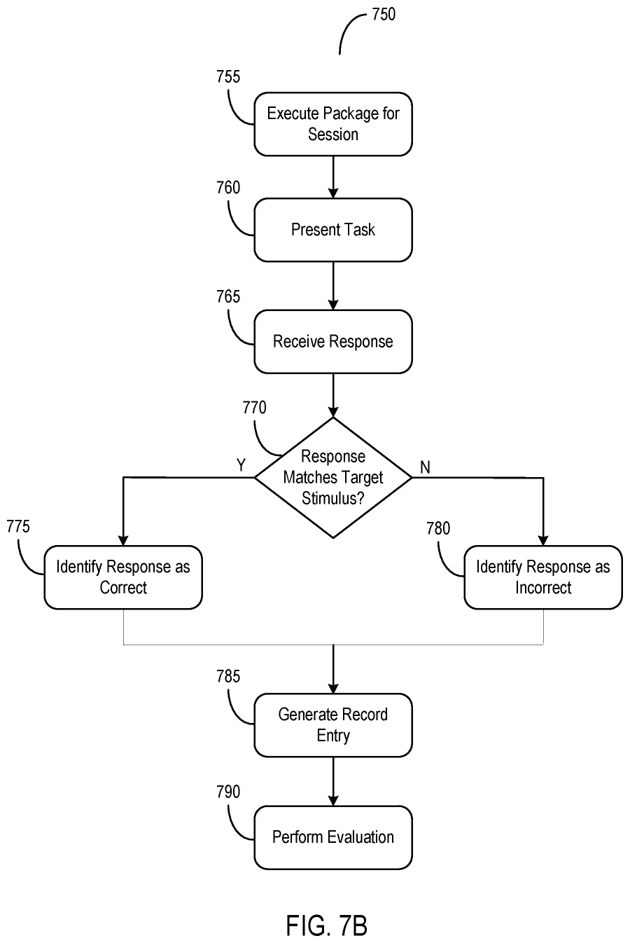

Referring now to FIG. 7A, depicted is a flow diagram of a method 700 of associating stimuli with users to address conditions. The method 700 may be implemented using any of the components as detailed herein in conjunction with FIG. 1-3 or 8. Under method 700, a computing system (e.g., the session configuration system 105 or the user device 110) may present a stimulus (e.g., the stimulus 195) to a user (e.g., the user 210) (705). The computing system may identify a relevance value of the stimulus with a condition of the user (710). The computing system may determine whether the relevance value satisfies a threshold (715). If the relevance value does not satisfy the threshold, the computing system may classify the stimulus as not non-neutral (720). On the other hand, if the relevance value does satisfy the threshold, the computing system may classify the stimulus as non-neutral (725). The computing system may identify a neutral stimulus (e.g., the neutral stimulus 190) to associate (or pair) with the non-neutral stimulus (e.g., the non-neutral stimulus 190′) (730). The computing system may store an association of the non-neutral stimulus and the neutral stimulus with the user (735). The computing system may generate a package (e.g., the session package 235) for a session to address the condition of the user (740).

Referring now to FIG. 7B, depicted is a flow diagram of a method 750 of performing sessions to provide stimuli to users to address conditions. The method 750 may be implemented using any of the components as detailed herein in conjunction with FIG. 1-3 or 8. The method 750 may be performed prior to, in conjunction with, or subsequent to the method 700. Under method 750, a computing system (e.g., the session configuration system 105 or the user device 110) may execute the package for the session to address the condition of the user (755). The computing system may present a task with the stimuli and a cue (760). The computing system may receive a response from the user indicating a selection of one of the stimuli (765). The computing system may determine whether the selection in the response matches a target stimulus (770). If the response is determined to match the target stimulus, the computing system may identify the response as correct (775). In contrast, if the response is determined not to match the target stimulus, the computing system may identify the response as incorrect (780). The computing system may generate a record entry based on the response (785). The computing system may perform an evaluation of the user with respect to the condition to be addressed (790).

### B. Network and Computing Environment

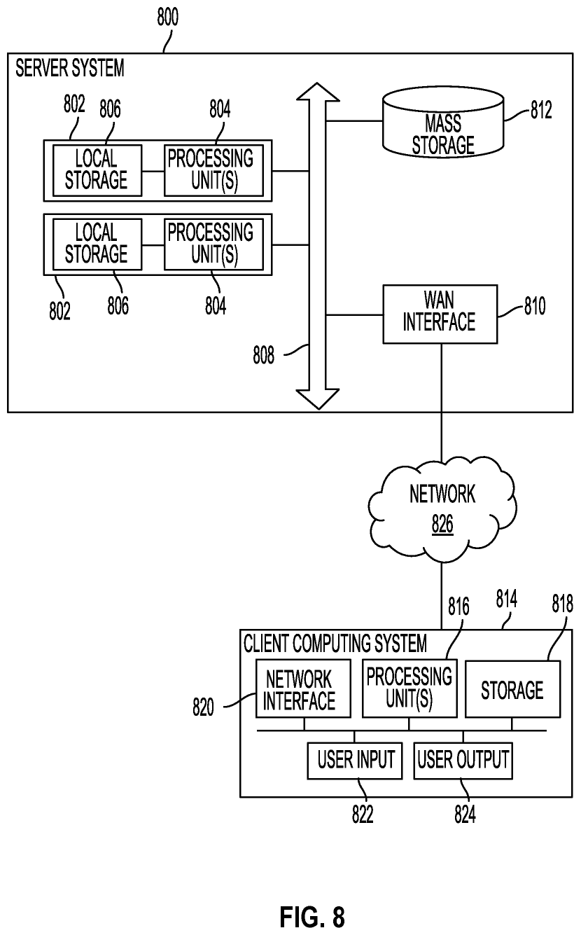

Various operations described herein can be implemented on computer systems. FIG. 8 shows a simplified block diagram of a representative server system 800, client computer system 814, and network 826 usable to implement certain embodiments of the present disclosure. In various embodiments, server system 800 or similar systems can implement services or servers described herein or portions thereof. Client computer system 814 or similar systems can implement clients described herein. The system 100 described herein can be similar to the server system 800. Server system 800 can have a modular design that incorporates a number of modules 802 (e.g., blades in a blade server embodiment); while two modules 802 are shown, any number can be provided. Each module 802 can include processing unit(s) 804 and local storage 806.

Processing unit(s) 804 can include a single processor, which can have one or more cores, or multiple processors. In some embodiments, processing unit(s) 804 can include a general-purpose primary processor as well as one or more special-purpose co-processors such as graphics processors, digital signal processors, or the like. In some embodiments, some or all processing units 804 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 804 can execute instructions stored in local storage 806. Any type of processors in any combination can be included in processing unit(s) 804.

Local storage 806 can include volatile storage media (e.g., DRAM, SRAM, SDRAM, or the like) and/or non-volatile storage media (e.g., magnetic or optical disk, flash memory, or the like). Storage media incorporated in local storage 806 can be fixed, removable, or upgradeable as desired. Local storage 806 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 804 need at runtime. The ROM can store static data and instructions that are needed by processing unit(s) 804. The permanent storage device can be a non-volatile read-and-write memory device that can store instructions and data even when module 802 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 806 can store one or more software programs to be executed by processing unit(s) 804, such as an operating system and/or programs implementing various server functions such as functions of the system 100 or any other system described herein, or any other server(s) associated with system 100 or any other system described herein.

“Software” refers generally to sequences of instructions that, when executed by processing unit(s) 804, cause server system 800 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 804. Software can be implemented as a single program or a collection of separate programs or program modules that interact as desired. From local storage 806 (or non-local storage described below), processing unit(s) 804 can retrieve program instructions to execute and data to process in order to execute various operations described above.

In some server systems 800, multiple modules 802 can be interconnected via a bus or other interconnect 808, forming a local area network that supports communication between modules 802 and other components of server system 800. Interconnect 808 can be implemented using various technologies, including server racks, hubs, routers, etc.

A wide area network (WAN) interface 810 can provide data communication capability between the local area network (e.g., through the interconnect 808) and the network 826, such as the Internet. Other technologies can be used to communicatively couple the server system with the network 826, including wired (e.g., Ethernet, IEEE 802.3 standards) and/or wireless technologies (e.g., Wi-Fi, IEEE 802.11 standards).

In some embodiments, local storage 806 is intended to provide working memory for processing unit(s) 804, providing fast access to programs and/or data to be processed while reducing traffic on interconnect 808. Storage for larger quantities of data can be provided on the local area network by one or more mass storage subsystems 812 that can be connected to interconnect 808. Mass storage subsystem 812 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 812. In some embodiments, additional data storage resources may be accessible via WAN interface 810 (potentially with increased latency).

Server system 800 can operate in response to requests received via WAN interface 810. For example, one of modules 802 can implement a supervisory function and assign discrete tasks to other modules 802 in response to received requests. Work allocation techniques can be used. As requests are processed, results can be returned to the requester via WAN interface 810. Such operations can generally be automated. Further, in some embodiments, WAN interface 810 can connect multiple server systems 800 to each other, providing scalable systems capable of managing high volumes of activity. Other techniques for managing server systems and server farms (collections of server systems that cooperate) can be used, including dynamic resource allocation and reallocation.

Server system 800 can interact with various user-owned or user-operated devices via a wide-area network such as the Internet. An example of a user-operated device is shown in FIG. 8 as client computing system 814. Client computing system 814 can be implemented, for example, as a consumer device such as a smartphone, other mobile phone, tablet computer, wearable computing device (e.g., smart watch, eyeglasses), desktop computer, laptop computer, and so on.

For example, client computing system 814 can communicate via WAN interface 810. Client computing system 814 can include computer components such as processing unit(s) 816, storage device 818, network interface 820, user input device 822, and user output device 824. Client computing system 814 can be a computing device implemented in a variety of form factors, such as a desktop computer, laptop computer, tablet computer, smartphone, other mobile computing device, wearable computing device, or the like.

Processing unit(s) 816 and storage device 818 can be similar to processing unit(s) 804 and local storage 806 described above. Suitable devices can be selected based on the demands to be placed on client computing system 814; for example, client computing system 814 can be implemented as a “thin” client with limited processing capability or as a high-powered computing device. Client computing system 814 can be provisioned with program code executable by processing unit(s) 816 to enable various interactions with server system 800.

Network interface 820 can provide a connection to the network 826, such as a wide area network (e.g., the Internet) to which WAN interface 810 of server system 800 is also connected. In various embodiments, network interface 820 can include a wired interface (e.g., Ethernet) and/or a wireless interface implementing various RF data communication standards such as Wi-Fi, Bluetooth, or cellular data network standards (e.g., 3G, 5G, LTE, etc.).

User input device 822 can include any device (or devices) via which a user can provide signals to client computing system 814; client computing system 814 can interpret the signals as indicative of user requests or information. In various embodiments, user input device 822 can include any or all of a keyboard, touch pad, touch screen, mouse or other pointing device, scroll wheel, click wheel, dial, button, switch, keypad, microphone, and so on.

User output device 824 can include any device via which client computing system 814 can provide information to a user. For example, user output device 824 can include display-to-display images generated by or delivered to client computing system 814. The display can incorporate various image generation technologies, e.g., a liquid crystal display (LCD), light-emitting diode (LED) display including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 824 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 804 and 816 can provide various functionality for server system 800 and client computing system 814, including any of the functionality described herein as being performed by a server or client, or other functionality.

It will be appreciated that server system 800 and client computing system 814 are illustrative and that variations and modifications are possible. Computer systems used in connection with embodiments of the present disclosure can have other capabilities not specifically described here. Further, while server system 800 and client computing system 814 are described with reference to particular blocks, it is to be understood that these blocks are defined for convenience of description and are not intended to imply a particular physical arrangement of component parts. For instance, different blocks can but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may refer to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of associating stimuli with conditions of users, comprising:
identifying, by a computing system, in response to presenting a first stimulus to a user, a relevance value of the first stimulus indicative of a relevance of the first stimulus with a condition of the user;
classifying, by the computing system, responsive to the relevance value satisfying a threshold, the first stimulus as having a non-neutral reaction type associated with the condition to the user;
storing, by the computing system, in one or more data structures, an association between the user and the first stimulus classified as having the non-neutral reaction type for presenting in a therapy session to address the condition of the user; and
providing, by the computing system, instructions for presenting the first stimulus in the therapy session to address the condition of the user.

2. The method of claim 1, further comprising:
selecting, by the computing system, from a plurality of stimuli classified as having a neutral reaction type, a second stimulus using a characteristic of the first stimulus;
generating, by the computing system, a set of stimuli comprising the first stimulus and the second stimulus; and
wherein storing the association further comprises storing the association between the user and the set of stimuli for presenting both the first stimulus and the second stimulus in the therapy session for addressing the condition of the user.

3. The method of claim 2, further comprising:
identifying, by the computing system, from the one or more data structures, the association between a user and a set of stimuli comprising (i) the first stimulus and (ii) a second stimulus having a neutral reaction type;
presenting, by the computing system, in the therapy session for the user, the first stimulus and the second stimulus, concurrent with a cue to increase a likelihood of selection of a stimulus from the set of stimuli having a target reaction type;
receiving, by the computing system, a response identifying a selection by the user of a stimulus from the set of stimuli.

4. The method of claim 2, wherein the therapy session includes an implicit association task (TAT), and further comprising:
presenting, by the computing system, the set of stimuli to increase the likelihood of selection of the stimulus having the neutral reaction type to associate the stimulus with a self of the user and to disassociate non-neutral stimuli from the self.

5. The method of claim 3, wherein the therapy session includes at least one of attention bias modification training (ABMT) or go/no-go training, and
wherein presenting further comprises presenting the cue to decrease the likelihood of selection of the stimulus having the non-neutral reaction type.

6. The method of claim 1, wherein the therapy session includes a personal trigger memory task, and
wherein presenting further comprises presenting, without a cue, a plurality of stimuli including (i) the first stimulus and (ii) a second stimulus having a neutral reaction type.

7. The method of claim 1, further comprising identifying, by the computing system, a plurality of delivery parameters for user based on response data to the therapy session.

8. The method of claim 1, further comprising identifying, by the computing system, the threshold to compare against the relevance value based on at least one of (i) a parameter of the therapy session or (ii) a relevance value of a second stimulus classified as having the non-neutral reaction type for the user.

9. The method of claim 1, wherein identifying the relevance value further comprises receiving a plurality of physiological measurements of the user in response to presentation of the first stimulus to the user.

10. The method of claim 1, further comprising:
identifying, by the computing system, a first evaluation dataset for the user at a first time prior to the therapy session and a second evaluation dataset for the user at a second time subsequent to the therapy session; and
determining, by the computing system, a progression for the user in addressing the condition based on the first evaluation dataset and the second evaluation dataset.

11. A system for associating stimuli with conditions of users, comprising:
a computing system having one or more processors coupled with memory, configured to:
identify, in response to presenting a first stimulus to a user, a relevance value of the first stimulus indicative of a relevance of the first stimulus with a condition of the user;
classify, responsive to the relevance value satisfying a threshold, the first stimulus as having a non-neutral reaction type associated with the condition to the user;
store, in one or more data structures, an association between the user and the first stimulus classified as having the non-neutral reaction type for presenting in a therapy session to address the condition of the user; and
provide instructions for presenting the first stimulus in the therapy session to address the condition of the user.

12. The system of claim 11, wherein the computing system is further configured to:
select, from a plurality of stimuli classified as having a neutral reaction type, a second stimulus using a characteristic of the first stimulus;
generate a set of stimuli comprising the first stimulus and the second stimulus; and
store the association between the user and the set of stimuli for presenting both the first stimulus and the second stimulus in the therapy session for addressing the condition of the user.

13. The system of claim 11, wherein the computing system is further configured to:
identify, from the one or more data structures, the association between a user and a set of stimuli comprising (i) the first stimulus and (ii) a second stimulus having a neutral reaction type;
present, in the therapy session for the user, the first stimulus and the second stimulus, concurrent with a cue to increase a likelihood of selection of a stimulus from the set of stimuli having a target reaction type;
receive a response identifying a selection by the user of a stimulus from the set of stimuli.

14. The system of claim 11, wherein the therapy session includes an implicit association task (TAT), and
wherein the computing system is further configured to present the pair of stimuli to increase the likelihood of selection of the stimulus having the neutral reaction type to associate the stimulus with a self of the user and to disassociate non-neutral stimuli from the self.

15. The system of claim 13, wherein the therapy session includes at least one of attention bias modification training (ABMT) or go/no-go training, and
wherein the computing system is further configured to present the cue to decrease the likelihood of selection of the stimulus having the non-neutral reaction type.

16. The system of claim 11, wherein the therapy session includes a personal trigger memory task, and
wherein the computing system is further configured to present, without a cue, a plurality of stimuli including (i) the first stimulus and (ii) a second stimulus having a neutral reaction type.

17. The system of claim 11, wherein the computing system is further configured to identify a plurality of delivery parameters for user based on response data to the therapy session.

18. The system of claim 11, wherein the computing system is further configured to identify the threshold to compare against the relevance value based on at least one of (i) a parameter of the therapy session or (ii) a relevance value of a second stimulus classified as having the non-neutral reaction type for the user.

19. The system of claim 11, wherein the computing system is further configured to receive a plurality of physiological measurements of the user in response to presentation of the first stimulus to the user.

20. The system of claim 11, wherein the computing system is further configured to:
identify a first evaluation dataset for the user at a first time prior to the therapy session and a second evaluation dataset for the user at a second time subsequent to the therapy session; and
determine a progression for the user in addressing the condition based on the first evaluation dataset and the second evaluation dataset.
